(19) United States
(12) Patent Application Publication
(10) Pub. No.: US 2019/0048335 A1
(43) Pub. Date: Feb. 14, 2019

(54) IMPROVED AMPLIFICATION AND SEQUENCING METHODS

(71) Applicant: LIFE TECHNOLOGIES CORPORATION, Carlsbad, CA (US)

(72) Inventors: Chieh-Yuan LI, Hayward, CA (US); David JOUN, San Francisco, CA (US); Shiaw-Min CHEN, San Jose, CA (US)

(21) Appl. No.: 16/085,815

(22) PCT Filed: Mar. 17, 2017
(86) PCT No.: PCT/US17/23019

§ 371 Date: Sep. 17, 2018

Related U.S. Application Data
(60) Provisional application No. 62/309,667, filed on Mar. 17, 2016.

(51) Int. Cl. C12N 15/10; C12Q 1/6806; C12N 15/11; C40B 20/04

(52) U.S. Cl. CPC C12N 15/1093; C12Q 1/6806; C12N 15/11; C40B 20/04; C12Q 2537/163; C12Q 2525/191; C12Q 2535/122; C12Q 2537/159; C12Q 2525/155

(57) ABSTRACT

In some embodiments, the disclosure relates generally to methods, as well as related systems, compositions, kits, and apparatuses for any one or any combination of: conducting a library preparation method which generates a mixture of desirable template polynucleotides and non-desirable polynucleotide byproducts, amplifying the resulting library, enriching the desirable template polynucleotides, and sequencing the enriched template polynucleotides. The methods, as well as related systems, compositions, kits, and apparatuses, of the present teachings can be used to improve sequencing data.

This application claims the benefit of priority under 35 U.S.C. § 119 to U.S. Provisional Application No. 62/309,667, filed Mar. 17, 2016; the disclosure of which is incorporated by reference in its entirety.

Throughout this application various publications, patents, and/or patent applications are referenced. The disclosures of the publications, patents and/or patent applications in their entireties are hereby incorporated by reference into this application in order to more fully describe the state of the art to which this invention pertains.

### SUMMARY

In some embodiments, the invention relates generally to methods, as well as related systems, compositions, kits, and apparatuses for enriching polynucleotides.

In one aspect, the invention provides methods, as well as related systems, compositions, kits, and apparatuses for producing a template polynucleotide attached to a support (e.g., bead or particle). In certain embodiments, provided is a method, comprising: (a) forming a reaction mixture by contacting: (i) a template polynucleotide, wherein the template polynucleotide comprises, 5′ to 3′, a second adaptor sequence, a template sequence, and a first adaptor sequence, (ii) at least one particle having a plurality of a capture primer attached thereon, wherein the capture primer is capable of hybridizing to the first adaptor sequence, (iii) a plurality of a solution-phase primer, wherein the solution phase primer comprises, 5′ to 3′, a fourth adaptor sequence and a third adaptor sequence, wherein the third adaptor sequence is capable of hybridizing to the complement of the second adaptor sequence; and (b) subjecting the reaction mixture to a nucleic acid amplification condition, thereby generating at least one particle attached to a polynucleotide population containing at least one polynucleotide that comprises, 5′ to 3′, the capture primer sequence, the complement of the template sequence, the complement of the third adaptor sequence, and the complement of the fourth adaptor sequence.

In some embodiments, the method further comprises contacting the at least one particle attached to a polynucleotide population with a sequencing primer, wherein the sequencing primer is capable of hybridizing to the complement of the third adaptor sequence. In some embodiments, the method comprises sequencing the polynucleotide population. In some embodiments, one or more wash steps can be performed after steps (a) and/or (b).

In some embodiments, the method further comprises contacting the at least one particle attached to a polynucleotide population with a blocking primer, wherein the blocking primer is capable of hybridizing to the complement of the fourth adaptor sequence. In some embodiments, the method further comprises: (a) contacting the at least one particle attached to a polynucleotide population with an enrichment primer, wherein the enrichment primer comprises a sequence that is capable of hybridizing to the complement of the third adaptor sequence, and wherein the enrichment primer comprises a first member of a binding pair, under conditions suitable for hybridizing the enrichment primer to the third adaptor sequence; (b) contacting the enrichment primer with a second member of the binding pair bound to a solid support; and (c) removing unbound components of the reaction mixture, thereby producing at least one particle attached to an enriched polynucleotide population.

In some embodiments, step (c) further comprises contacting the at least one particle attached to a polynucleotide population with a blocking primer, wherein the blocking primer is capable of hybridizing to the complement of the fourth adaptor sequence. In some embodiments, one or more wash steps can be performed after steps (a), (b) and/or (c).

In some embodiments, the method further comprises contacting the at least one particle attached to an enriched polynucleotide population with a sequencing primer, wherein the sequencing primer is capable of hybridizing to the complement of the third adaptor sequence. In some embodiments, the method comprises sequencing the polynucleotide population. In some embodiments, the method further comprises contacting the at least one particle attached to a polynucleotide population with a blocking primer, wherein the blocking primer is capable of hybridizing to the complement of the fourth adaptor sequence.

In some embodiments, the template polynucleotide is formed by fragmenting target polynucleotides isolated from a biological fluid, cell culture, or solid tissue. In some embodiments, one or more adaptors can be appended to the target polynucleotides using enzymatic ligation or primer extension. In some embodiments, the target polynucleotide is a non-fragmented polynucleotide. In some embodiments, the target polynucleotide is the result of an amplification reaction. In some embodiments, the target polynucleotides are isolated from a biological fluid and the biological fluid comprises blood, serum, plasma, saliva, sputum, sweat, tears, lavage fluid, amniotic fluid, cerebrospinal fluid, ascites, urine, or semen. In some embodiments, the attaching comprises ligation. In particular embodiments a first adaptor sequence and a second adaptor sequence are attached to a target polypeptide. In certain particular embodiments a first adaptor and a second adaptor are attached using a primer extension reaction. In certain particular embodiments a first and a second adaptor are attached by enzymatic ligation.

In some embodiments, the set of particles is a set of microparticles or a set of beads.

In some embodiments, the second adaptor sequence comprises at least one universal sequence. In some embodiments, the template polynucleotide further comprises at least one unique identifier sequence. In some embodiments, the at least one unique identifier sequence is located between the template sequence and the second adaptor sequence. In some embodiments, the at least one unique identifier sequence is located between the template sequence and the first adaptor sequence. In some embodiments, the at least one unique identifier sequence forms part of the first adaptor sequence or the second adaptor sequence. In some embodiments, the unique identifier sequence is a barcode sequence.

In some embodiments, the template sequence comprises 10 to 2000 base pairs, or 10 to 1500 base pairs, or 10 to 1000 base pairs, or 10 to 500 base pairs, or 50 to 500 base pairs of the target polynucleotide in its double-stranded form.

In some embodiments, the nucleic acid amplification condition comprises a primer extension reaction. In some embodiments, the nucleic acid amplification condition comprises an isothermal amplification condition or a thermocycling amplification condition. In some embodiments, the reaction mixture comprises a polymerase and a plurality of nucleotides. In some embodiments, the reaction mixture comprises a recombinase. In some embodiments, the recombinase is uvsX. In some embodiments, the reaction mixture comprises at least one recombinase accessory protein. In some embodiments, the reaction mixture comprises at least one recombinase loading protein and/or at least one single-stranded binding protein. In some embodiments, at least one recombinase loading protein is uvsY. In some embodiments, at least one single-stranded binding protein is gp32. In some embodiments, the recombinase (e.g., uvsX), recombinase loading protein (e.g., uvsY), and/or single-stranded binding protein (e.g., gp32) can be from a T4 bacteriophage.

In some embodiments, the reaction mixture is contained in a water droplet in an oil and water emulsion. In some embodiments, the reaction mixture is part of a single continuous liquid phase that does not provide compartmentalization. In some embodiments, the single continuous liquid phase lacks an oil and water emulsion. In some embodiments, the single continuous liquid phase is an aqueous phase.

In some embodiments, the method further comprises depositing at least one particle attached to a polynucleotide population or at least one particle attached to an enriched polynucleotide population on a support having at least one reaction site operatively coupled to a sensor. In some embodiments, the method further comprises depositing at least one particle attached to a polynucleotide population or at least one particle attached to an enriched polynucleotide population on a reaction site of a support having an array of reaction sites. In some embodiments, an array comprises a plurality of reaction sites. In some embodiments, particles attached to different polynucleotide populations are deposited on different reaction sites on the same array. In some embodiments, the different polynucleotides are sequenced in parallel at the different reaction sites of the array. In some embodiments, the individual reaction sites in the array are operatively coupled to at least one sensor.

In some embodiments, the method comprises sequencing the polynucleotide population, and wherein the sequencing is conducted at the reaction site. In some embodiments, the sequencing comprises detecting at least one nucleotide incorporation byproduct using the at least one sensor operatively coupled to the reaction site. In some embodiments, at least one nucleotide incorporation byproduct is selected from hydrogen ions, hydroxyl ions, pyrophosphate, charge transfer, and heat. In some embodiments, the sensor detects a change in pH. In some embodiments, the sensor comprises a field effect transistor (FET). In some embodiments, the sensor comprises a chemical field effect transistor (chemFET). In some embodiments, the sensor comprises an ion-sensitive field effect transistor (ISFET).

In some embodiments, methods of producing at least two sets of particles are provided, wherein a first set of particles comprises a first template sequence and a second set of particles comprises a second template sequence, wherein the first and second template sequences may be the same or different. In some embodiments, a method comprises: (a) forming a reaction mixture by contacting: (i) a first template polynucleotide, wherein the first template polynucleotide comprises, 5′ to 3′, a second adaptor sequence, a first identifier sequence, the first template sequence, and a first adaptor sequence, (ii) a second template polynucleotide, wherein the second template polynucleotide comprises, 5′ to 3′, a second adaptor sequence, a second identifier sequence, the second template sequence, and a third adaptor sequence, (iii) a first set of particles having a plurality of a first capture primer attached thereon, wherein the first capture primer is capable of hybridizing to the first adaptor sequence, (iv) a second set of particles having a plurality of a second capture primer attached thereon, wherein the second capture primer is capable of hybridizing to the third adaptor sequence, (v) a plurality of a solution-phase primer, wherein the solution phase primer comprises, 5′ to 3′, a fourth adaptor sequence and a fifth adaptor sequence, wherein the fifth adaptor sequence is capable of hybridizing to the complement of the second adaptor sequence; (b) subjecting the reaction mixture to a nucleic acid amplification condition, thereby generating: (i) a first set of particles attached to a first polynucleotide population containing at least one polynucleotide that comprises, 5′ to 3′, the first capture primer sequence, the complement of the first template sequence, the complement of the first identifier sequence, the complement of the fifth adaptor sequence, and the complement of the fourth adaptor sequence; and (ii) a second set of particles attached to a second polynucleotide population containing at least one polynucleotide that comprises, 5′ to 3′, the second capture primer sequence, the complement of the second template sequence, the complement of the second identifier sequence, the complement of the fifth adaptor sequence, and the complement of the fourth adaptor sequence.

In some embodiments, the first template polynucleotide comprises, 5′ to 3′, a second adaptor sequence, the first template sequence, a first identifier sequence, and a first adaptor sequence. In some embodiments, the second template polynucleotide comprises, 5′ to 3′, a second adaptor sequence, the second template sequence, a second identifier sequence, and a third adaptor sequence. Further, the first capture primer on the first set of particles may have the same or different sequence as the second capture primer on the second set of particles. The first and second identifier sequences may also have the same or different sequences.

In some embodiments, the method further comprises contacting the at least one particle attached to a polynucleotide population with a sequencing primer, wherein the sequencing primer is capable of hybridizing to the complement of the fifth adaptor sequence. In some embodiments, the method comprises sequencing the first polynucleotide population and/or the second polynucleotide population. In some embodiments, the method comprises sequencing the first polynucleotide population and the second polynucleotide population.

In some embodiments, the method further comprises contacting the first set of particles attached to a first polynucleotide population with a blocking primer, wherein the blocking primer is capable of hybridizing to the complement of the fourth adaptor sequence. In some embodiments, the method further comprises contacting the second set of particles attached to a second polynucleotide population with a blocking primer, wherein the blocking primer is capable of hybridizing to the complement of the fourth adaptor sequence. In some embodiments, one or more wash steps can be performed after steps (a) and/or (b).

In some embodiments, the method further comprises: (a) contacting the first set of particles attached to a first polynucleotide population with an enrichment primer, wherein the enrichment primer comprises a sequence that is capable of hybridizing to the complement of the fifth adaptor sequence, and wherein the enrichment primer comprises a first member of a binding pair, under conditions suitable for hybridizing the enrichment primer to the fifth adaptor sequence; (b) contacting the enrichment primer with a second member of the binding pair bound to a solid support; and (c) removing unbound components of the reaction mixture, thereby producing a first set of particles attached to a first enriched polynucleotide population.

In some embodiments, the method further comprises: (a) contacting the second set of particles attached to a second polynucleotide population with an enrichment primer, wherein the enrichment primer comprises a sequence that is capable of hybridizing to the complement of the fifth adaptor sequence, and wherein the enrichment primer comprises a first member of a binding pair, under conditions suitable for hybridizing the enrichment primer to the fifth adaptor sequence; (b) contacting the enrichment primer with a second member of the binding pair bound to a solid support; and (c) removing unbound components of the reaction mixture, thereby producing a second set of particles attached to a second enriched polynucleotide population.

In some embodiments, step (c) further comprises contacting the first set of particles attached to a first polynucleotide population and/or the second set of particles attached to a second polynucleotide population with a blocking primer, wherein the blocking primer is capable of hybridizing to the complement of the fourth adaptor sequence.

In some embodiments, the method further comprises contacting the first set of particles attached to a first enriched polynucleotide population and/or the second set of particles attached to a second enriched polynucleotide population with a sequencing primer, wherein the sequencing primer is capable of hybridizing to the complement of the fifth adaptor sequence. In some embodiments, the method comprises sequencing the first enriched polynucleotide population and/or the second enriched polynucleotide population. In some embodiments, the method further comprises contacting the first set of particles attached to a first enriched polynucleotide population with a blocking primer, wherein the blocking primer is capable of hybridizing to the complement of the fourth adaptor sequence. In some embodiments, the method further comprises contacting the second set of particles attached to a second enriched polynucleotide population with a blocking primer, wherein the blocking primer is capable of hybridizing to the complement of the fourth adaptor sequence. In some embodiments, one or more wash steps can be performed after steps (a), (b) and/or (c).

In some embodiments, the first template polynucleotide is formed by fragmenting target polynucleotides isolated from a biological fluid, cell culture, or solid tissue, and attaching a second adaptor sequence, a first identifier sequence, and a first adaptor sequence to the fragmented target polynucleotides. In some embodiments, the first template polynucleotide is a non-fragmented polynucleotide. In some embodiments, the first template polynucleotide is the result of an amplification reaction. In some embodiments, the second adaptor sequence and the first identifier sequence are part of the same oligonucleotide being attached to fragmented target polynucleotides. In some embodiments, the second template polynucleotide is formed by fragmenting target polynucleotides isolated from a biological fluid, cell culture, or solid tissue, and attaching a second adaptor sequence, a second identifier sequence, and a third adaptor sequence to the fragmented target polynucleotides. In some embodiments, the second template polynucleotide is a non-fragmented polynucleotide. In some embodiments, the second template polynucleotide is the result of an amplification reaction. In some embodiments, the second adaptor sequence and the second identifier sequence are part of the same oligonucleotide being attached to fragmented target polynucleotides.

In some embodiments, the target polynucleotides are isolated from a biological fluid and the biological fluid comprises blood, serum, plasma, saliva, sputum, sweat, tears, lavage fluid, amniotic fluid, cerebrospinal fluid, ascites, urine, or semen. In some embodiments, the attaching comprises ligation. In some embodiments, the adaptors and/or identifier sequences are attached using a primer extension reaction.

In some embodiments, the first set of particles and the second set of particles are microparticles or beads. In some embodiments, the second adaptor sequence comprises at least one universal sequence. In some embodiments, the first identifier sequence is a first barcode sequence and the second identifier sequence is a second barcode sequence. In some embodiments, the first and second barcode sequences can be the same or different.

In some embodiments, each template sequence comprises 10 to 2000 base pairs, or 10 to 1500 base pairs, or 10 to 1000 base pairs, or 10 to 500 base pairs, or 50 to 500 base pairs of the target polynucleotide in its double-stranded form.

In some embodiments, the nucleic acid amplification condition comprises a primer extension reaction. In some embodiments, the nucleic acid amplification condition comprises an isothermal amplification condition or a thermocycling amplification condition. In some embodiments, the reaction mixture comprises a polymerase and a plurality of nucleotides. In some embodiments, the reaction mixture comprises a recombinase. In some embodiments, the recombinase is uvsX. In some embodiments, the reaction mixture comprises at least one recombinase accessory protein. In some embodiments, the reaction mixture comprises at least one recombinase loading protein and/or at least one single-stranded binding protein. In some embodiments, at least one recombinase loading protein is uvsY. In some embodiments, at least one single-stranded binding protein is gp32.

In some embodiments, the reaction mixture is contained in a water droplet in an oil and water emulsion. In some embodiments, the reaction mixture is part of a single continuous liquid phase that does not provide compartmentalization. In some embodiments, the single continuous liquid phase lacks an oil and water emulsion. In some embodiments, the single continuous liquid phase is an aqueous phase.

In some embodiments, the method further comprises depositing the first set of particles attached to a first polynucleotide population or a first set of particles attached to a first enriched polynucleotide population on a first reaction site of a support having an array of reaction sites. In some embodiments, the method further comprises depositing the second set of particles attached to a second polynucleotide population or a second set of particles attached to a second enriched polynucleotide population on a second reaction site of a support having an array of reaction sites. In some embodiments, the first and second set of particles (both sets being attached to a first and second polynucleotide population, respectively) can be deposited onto the same array of reaction sites, or can be deposited onto different arrays where both arrays have a plurality of reaction sites. In some embodiments, the individual reaction sites in the array are operatively coupled to at least one sensor. In some embodiments, the method comprises sequencing the first polynucleotide population or the first enriched polynucleotide population, and wherein the sequencing is conducted at the first reaction site. In some embodiments, the method comprises sequencing the second polynucleotide population or the second enriched polynucleotide population, and wherein the sequencing is conducted at the second reaction site. In some embodiments, the sequencing comprises detecting at least one nucleotide incorporation byproduct using the at least one sensor operatively coupled to the first reaction site and/or the second reaction site. In some embodiments, at least one nucleotide incorporation byproduct is selected from hydrogen ions, hydroxyl ions, pyrophosphate, charge transfer, and heat. In some embodiments, the sensor detects a change in pH. In some embodiments, the sensor comprises a field effect transistor (FET). In some embodiments, the sensor comprises a chemical field effect transistor (chemFET). In some embodiments, the sensor comprises an ion-sensitive field effect transistor (ISFET).

### DESCRIPTION OF THE DRAWINGS

FIG. 1 is a schematic that shows how a standard library preparation and bead templating methodologies can lead to the generation of primer dimers that interfere with the sequencing data.

FIG. 2 is a schematic that provides a comparison of the standard method of sequencing templated beads (i.e., “Standard Method”) versus sequential primer sequencing.

FIG. 3 is a schematic that illustrates a method for sequencing bead-template polynucleotides.

FIG. 4 is a schematic that illustrates a method for sequencing bead-template polynucleotides.

FIG. 5 is a schematic that illustrates a method for sequencing bead-template polynucleotides.

FIG. 6 is a schematic that illustrates a method for sequencing bead-template polynucleotides.

FIG. 7A shows sequencing filter and trim data results from the experiments described in Example 1 using the standard library prep and bead templating workflow.

FIG. 7B shows sequencing filter and trim data results from the experiments described in Example 1 using the Sequential Primer library prep and bead templating workflow.

FIG. 8 shows the error rate per cycle results from the experiments described in Example 1 using the standard library prep and bead templating workflows, and error rates of each base at each flow of nucleotides over 400 nucleotide flows.

FIG. 8B shows the error rate per cycle results from the experiments described in Example 1 using the standard library prep and bead templating workflows, and error rates of each base at each flow of nucleotides over 100 nucleotide flows.

FIG. 8C shows the error rate per cycle results from the experiments described in Example 1 using the Sequential Primer library prep and bead templating workflows, and error rates of each base at each flow of nucleotides over 400 nucleotide flows.

FIG. 8D shows the error rate per cycle results from the experiments described in Example 1 using the Sequential Primer library prep and bead templating workflows, and error rates of each base at each flow of nucleotides over 100 nucleotide flows.

FIG. 9 is a schematic that shows an enrichment workflow using enrichment primers and optionally blocker primers.

### DETAILED DESCRIPTION

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

As used herein the terms “amplify”, “amplifying”, “amplification” and other related terms include producing multiple copies of an original biomolecule. In some embodiments, nucleic acid amplification produces multiple copies of an original polynucleotide (e.g., target polynucleotide), where the copies comprise a template sequence, or the copies comprise a sequence that is substantially identical to a template sequence.

As used herein the terms “hybridize”, “hybridizing”, “hybridization” and other related terms include hydrogen bonding between two different nucleic acids, or between two different regions of a nucleic acid, to form a duplex nucleic acid. Hybridization can comprise Watson-Crick or Hoogstein binding to form a duplex nucleic acid. The two different nucleic acids, or the two different regions of a nucleic acid, may be complementary, or partially complementary. The complementary base pairing can be the standard A-T or C-G base pairing, or can be other forms of base-pairing interactions. Duplex nucleic acids can include mismatched base-paired nucleotides. Complementary nucleic acid strands need not hybridize with each other across their entire length.

In some embodiments, conditions that are suitable for nucleic acid hybridization and/or for washing conditions include parameters such as salts, buffers, pH, temperature, GC % content of the polynucleotide and primers, and/or time. For example, conditions suitable for hybridizing or washing nucleic acids (e.g., polynucleotides and primers) can include hybridization solutions having sodium salts, such as NaCl, sodium citrate and/or sodium phosphate. In some embodiments, hybridization or wash solutions can include formamide (e.g., about 10-75%) and/or sodium dodecyl sulfate (SDS) (e.g., about 0.01-0.7%). In some embodiments, a hybridization solution can be a stringent hybridization solution which can include any combination of formamide (e.g., about 50%), 5×SSC (e.g., about 0.75 M NaCl and about 0.075 M sodium citrate), sodium phosphate (e.g., about 50 mM at about pH 6.8), sodium pyrophosphate (e.g., about 0.1%), 5×Denhardt's solution, SDS (e.g., about 0.1%), and/or dextran sulfate (e.g., about 10%). In some embodiments, the hybridization or washing solution can include BSA (bovine serum albumin). In some embodiments, hybridization or washing can be conducted at a temperature range of about 15-25° C., or about 25-35° C., or about 35-45° C., or about 45-55° C., or about 55-65° C., or about 65-75° C., or about 75-85° C., or about 85-95° C., or about 95-99° C., or higher.

In some embodiments, hybridization or washing can be conducted for a time range of about 1-10 minutes, or about 10-20 minutes, or about 20-30 minutes, or about 30-40 minutes, or about 40-50 minutes, or about 50-60 minutes, or longer.

In some embodiments, hybridization or wash conditions can be conducted at a pH range of about 5-10, or about pH 6-9, or about pH 6.5-8, or about pH 6.5-7.

Methods for nucleic acid hybridization and washing are well known in the art. For example, thermal melting temperature (Tₘ) for nucleic acids can be a temperature at which half of the nucleic acid strands are double-stranded and half are single-stranded under a defined condition. In some embodiments, a defined condition can include ionic strength and pH in an aqueous reaction condition. A defined condition can be modulated by altering the concentration of salts (e.g., sodium), temperature, pH, buffers, and/or formamide. Typically, the calculated thermal melting temperature can be at about 5-30° C. below the Tₘ, or about 5-25° C. below the Tₘ, or about 5-20° C. below the Tₘ, or about 5-15° C. below the Tₘ, or about 5-10° C. below the Tₘ. Methods for calculating a Tₘ are well known and can be found in Sambrook (1989 in “Molecular Cloning: A Laboratory Manual”, 2^{nd} edition, volumes 1-3; Wetmur 1966, J. Mol. Biol., 31:349-370; Wetmur 1991 Critical Reviews in Biochemistry and Molecular Biology, 26:227-259). Other sources for calculating a Tₘ for hybridizing or denaturing nucleic acids include OligoAnalyze (from Integrated DNA Technologies) and Primer3 (distributed by the Whitehead Institute for Biomedical Research).

In some embodiments, the term “surface” can be an outer or top-most layer or boundary of an object or a support. In some embodiments, a surface can be interior to the boundary of an object.

In some embodiments, a surface or support can be a planar surface, as well as concave, convex, or any combination thereof. In some embodiments, a surface or support can be a bead, particle, microparticle, sphere, filter, flowcell, well, microwell, groove, channel reservoir, gel or inner wall of a capillary. In some embodiments, a surface or support includes the inner walls of a capillary, a channel, a well, microwell, groove, channel, reservoir. In some embodiments, a surface or support can include texture (e.g., etched, cavitated, pores, three-dimensional scaffolds or bumps). In some embodiments, a surface or support can be porous, semi-porous or non-porous.

In some embodiments, particles or beads can have a shape that is spherical, hemispherical, cylindrical, barrel-shaped, toroidal, rod-like, disc-like, conical, triangular, cubical, polygonal, tubular, wire-like or irregular.

In some embodiments, a surface or support can be made from any material, including glass, borosilicate glass, silica, quartz, fused quartz, mica, polyacrylamide, plastic polystyrene, polycarbonate, polymethacrylate (PMA), polymethyl methacrylate (PMMA), polydimethylsiloxane (PDMS), silicon, germanium, graphite, ceramics, silicon, semiconductor, high refractive index dielectrics, crystals, gels, polymers, or films (e.g., films of gold, silver, aluminum, or diamond).

In some embodiments, a surface or support can be magnetic or paramagnetic. In some embodiments, a surface or support can be paramagnetic beads (particle) attached with streptavidin, for example Dynabeads™ M-270 (from Invitrogen, Carlsbad, Calif.). A bead or particle can have an iron core, or comprise a hydrogel or agarose (e.g., Sepharose™).

In some embodiments, the surface or support (including interior scaffolds of a bead or particle) can be attached with a plurality of a capture primer. A surface or support can be coated with an acrylamide, carboxylic or amine compound for attaching a nucleic acid (e.g., a capture primer). In some embodiments, an amino-modified nucleic acid (e.g., primer) can be attached to a surface that is coated with a carboxylic acid. In some embodiments, an amino-modified nucleic acid can be reacted with ethyl (dimethylaminopropyl) carbodiimide (EDC) or EDAC for attachment to a carboxylic acid coated surface (with or without N-hydoxysuccinimide (NETS)). A capture primer can be immobilized to an acrylamide compound coating on a surface. The particles can be coated with an avidin-like compound (e.g., streptavidin) for binding biotinylated nucleic acids.

As used herein, a population of nucleic acids or polynucleotides is considered to be substantially “monoclonal” or is considered to have substantial “monoclonality” if a substantial portion of its members have substantially identical sequence. In some embodiments, members of a population need not be 100% identical, for example a certain number of “errors” may occur during the course of nucleic acid amplification reactions. In some embodiments, at least 50% of the members of a population are at least 90% identical to a reference nucleic acid molecule (i.e., a nucleic acid of defined sequence used as a basis for a sequence comparison). In some embodiments, at least 50%, at least 60%, at least 70%, at least 80%, at least 90%, at least 95%, at least 99%, or more of the members of a population comprise a substantially identical sequence compared to a reference nucleic acid molecule. In some embodiments, a low or insubstantial level of mixing of non-homologous nucleic acids may occur during nucleic acid amplification reactions described herein, and thus a clonal population may contain a minority of diverse nucleic acids (e.g., less than about 30%, less than about 20%, less than about 10%, less than about 5%, or less than about 1%, or less than about 0.1%).

For example, a nucleic acid amplification reaction can generate amplified polynucleotide strands having a sequence that is complementary to a template polynucleotide or having the same sequence as the template polynucleotide. In some embodiments the population of amplified polynucleotide strands is substantially monoclonal. A population of amplified polynucleotide strands is considered to be substantially monoclonal if a substantial portion of its members have a sequence that is substantially identical to a sequence that is complementary to the template sequence or have a sequence that is substantially identical to the template sequence.

Recent advances in nucleic acid sequencing technology have reduced the cost and time necessary to obtain sequence data from a sample. Accordingly, sequence data is increasingly acquired and used in clinical and field applications, including personalized medicine and detection and identification of pathogens and other organisms.

Various massively parallel sequencing platforms, such as the Ion Torrent sequencing platform, involve amplification of a template sequence and creation of “monoclonal” templated particles, comprising multiple copies of the same sequence. Current amplification methods include, but are not limited to, emulsion-based PCR (emPCR) and recombinase-mediated amplification (RPA). Both methods involve the use of a distal solution-phase oligonucleotide and an oligonucleotide covalently linked to the particle to act as primers for template amplification. Primer-based amplification systems run the risk of primer dimer formation. Primer dimer formation may be caused, for example, by primer-to-primer interaction, for example, due to partial sequence homology, and creation of an extendable 5′ overhang, which may form a site for polymerase recognition and extension. To avoid formation of primer dimers, common approaches include, for example, particular methods of primer design (in silico, blast, consideration of secondary structure, use of modified primers, etc.), and reaction condition optimization (adjustments to primer purity, primer concentration, salt concentration, additives, temperature, etc).

There is still a need for greater speed and accuracy in obtaining amplification and sequence data, including sequence data for specific loci, e.g., loci comprising polymorphisms such as single nucleotide polymorphisms or signature nucleotides that identify a particular strain, species, or other taxon, within complex samples such as genomic DNA and total RNA.

Provided herein are methods, compositions, and systems that can solve this need and/or provide other benefits. In some embodiments, methods, compositions, and systems are provided in which the formation of primer dimers and other byproducts during particle templating is reduced. In some embodiments, the methods, as well as related systems, compositions, kits, and apparatuses involve the use of an enrichment primer, for example, to select for template polynucleotides, or to counter-select primer dimers. In some embodiments, the methods, as well as related systems, compositions, kits, and apparatuses involve the use of an enrichment primer, for example, to select for particles comprising template polynucleotides and/or to counter-select particles comprising primer dimers. By reducing or removing primer dimers, the signal to noise ratio in a sequencing reaction may be increased, allowing greater sequence throughput and accuracy.

A number of improvements may be possible with the present methods versus the standard method. There may be an increase in total sequencing throughput as throughput is not eaten up by primer dimers. There may be an increase in signal-to-noise ratio (SNR) because the system has fewer contaminant primer dimers; this advantage is especially useful for diagnostic purposes. There may also be increased sensitivity and accuracy of the data.

A polynucleotide library preparation workflow produces a mixture of desirable template polynucleotides and non-desirable polynucleotide byproducts. For example, a library preparation workflow can be conducted using ligation and/or primer extension to append one or more adaptor sequences to the ends of a plurality of initial polynucleotides to generate a plurality of desirable template polynucleotides, however, results may inevitably generate a plurality of non-desirable byproducts, where the undesirable byproducts comprise, e.g., adaptor-dimers, primer-dimers.

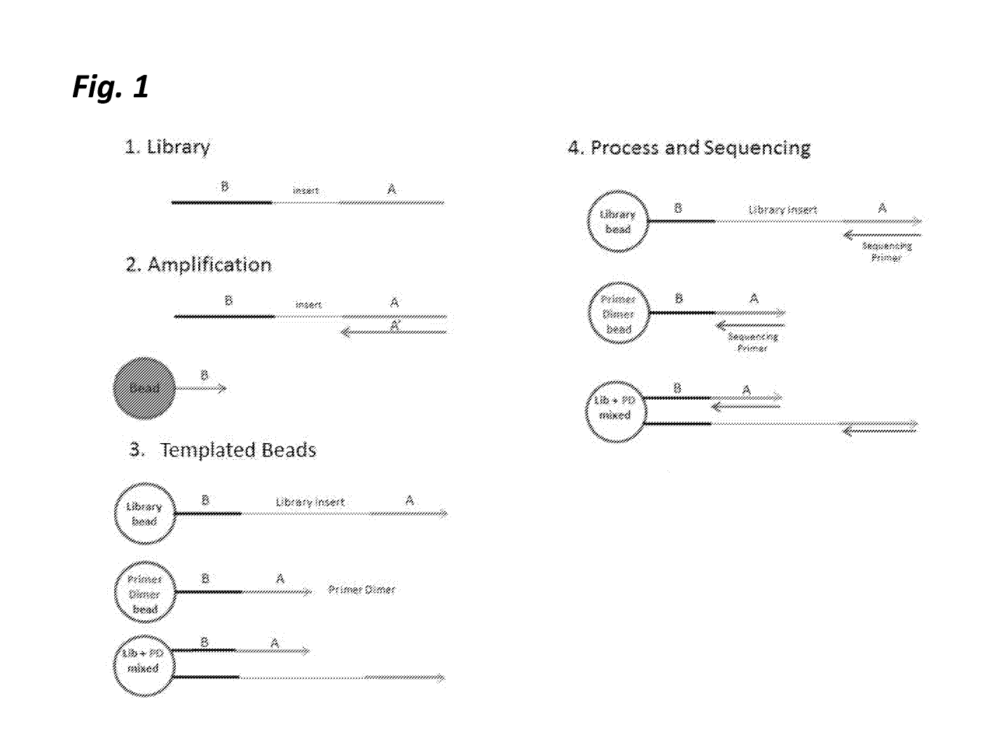

A standard library preparation and bead-templating workflow can result in production of a mixture of desirable library beads and undesirable bead primer-dimer byproducts. FIG. 1 depicts a schematic showing a standard workflow comprising a library of DNA molecules, wherein individual molecules include an insert fragment flanked by A and B adaptors (Step 1 Library). The library molecules are amplified using a solution-phase primer (A′ primer) to generate amplicon library molecules having a complement sequence of the insert fragment flanked by complement sequences of the A and B adaptors (Step 2 Amplification). The library molecules are clonally amplified onto beads that carry a plurality of B capture primers (Step 2 Amplification) which generates various types of templated beads (Step 3 Templated Beads). B capture primers can hybridize to the complement of B adaptors of the amplicon library molecules. Templated beads include desirable library beads, each having a bead attached to substantially monoclonal copies of a library molecule, where library molecules include a library insert sequence flanked by A and B adaptor sequences, where a B adaptor sequence is attached to the bead (Step 3 Templated Beads). Templated beads also include undesirable primer-dimer-beads (also known as PD), which include multiple copies of A-B primer dimers attached to beads (Step 3 Templated Beads), where a B adaptor sequence is attached to the bead. Template beads also can include undesirable mixed library-primer-dimer beads, each having copies of A-B primer dimers and library molecules attached to a same bead (Step 3 Templated Beads), where the library molecules include a library insert flanked by an A and B adaptor. Templated beads are loaded an array of wells on a sequencing chip and then sequenced using a sequencing primer that anneals to the A adaptor sequence on the various types of templated beads (Step 4 Process and Sequencing). The sequencing data from the library beads result in a good signal and accurate reads. By contrast, primer dimer beads do not yield relevant data and reduce sequencing throughput. The library plus primer dimer bead also can result in yield of low quality data with low signal and poor signal-to-noise ratio (SNR).

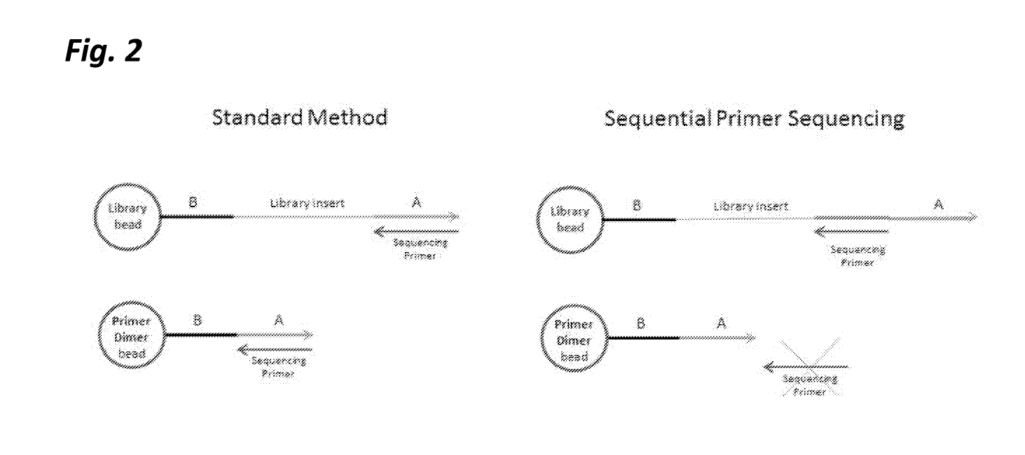

In a sequential primer sequencing methods that are described according to the present teachings, a modified library preparation workflow generates various types of templated beads, including: primer-dimer beads, mixed library-primer-dimer beads (not shown), and library beads (FIG. 2 Sequential Primer Sequencing). The primer-dimer-bead includes a plurality of A-B primer-dimers attached to a bead. The library bead is attached with substantially monoclonal copies of library molecules having an insert sequence flanked by a B adaptor and an additional adaptor sequence that is positioned 5′ to the A adaptor sequence (FIG. 2 Sequential Primer Sequencing). The mixed library-primer-dimer bead includes a plurality of A-B primer-dimers and library molecules attached thereon, where the library molecules include an insert sequence flanked by a B adaptor and an additional adaptor sequence that is positioned 5′ to the A adaptor sequence (not shown). In the sequential primer sequencing method, the sequencing primer anneals to a sequence within the additional adaptor sequence which is located outside of adaptor A (FIG. 2 Sequential Primer Sequencing). In the sequential primer sequencing method, the sequencing primer does not anneal to the A adaptor sequence on the primer-dimer-beads, and the primer-dimers are not sequenced. In sequential primer sequencing, library molecules having the additional adaptor sequence which hybridizes to the sequencing primers and is located outside of the A adaptor, can be generated by a number of different means that are described throughout this application. The sequential primer sequencing method uses an alternative sequencing primer (or enrichment primer with biotin) that is distinct from the templating primer and that is not complementary to a sequence within an adaptor. Therefore, the sequential primer sequencing method may differentiate the unwanted side product (i.e., amplification from the primer dimer bead) from the product of interest amplified from a library insert. Thus, beads including primer dimer beads are not sequenced using the sequential primer sequencing methodology.

In some embodiments, a Sequential Primer Sequencing method includes: depositing to a reaction site on a support, at least one bead attached to polynucleotides, wherein the support contains plurality of reaction sites arranged in an array. In some embodiments, beads that are deposited include primer-dimer beads, mixed library-primer-dimer beads, and library beads (FIG. 2). In some embodiments, at least one reaction site is operatively coupled to a sensor. In some embodiments, beads attached to different polynucleotide populations are deposited on different reaction sites on the same array. In some embodiments, the different polynucleotides are sequenced in parallel at the different reaction sites of the array. In some embodiments, the individual reaction sites in the array are operatively coupled to at least one sensor.

In some embodiments, the Sequential Primer Sequencing method (FIG. 2) comprises sequencing the library molecules attached to the beads, and wherein the sequencing is conducted at the reaction site. In some embodiments, the sequencing comprises detecting at least one nucleotide incorporation byproduct using the at least one sensor operatively coupled to the reaction site. In some embodiments, at least one nucleotide incorporation byproduct is selected from hydrogen ions, hydroxyl ions, pyrophosphate, charge transfer, and heat. In some embodiments, the sensor detects a change in pH. In some embodiments, the sensor comprises a field effect transistor (FET). In some embodiments, the sensor comprises a chemical field effect transistor (chemFET). In some embodiments, the sensor comprises an ion-sensitive field effect transistor (ISFET).

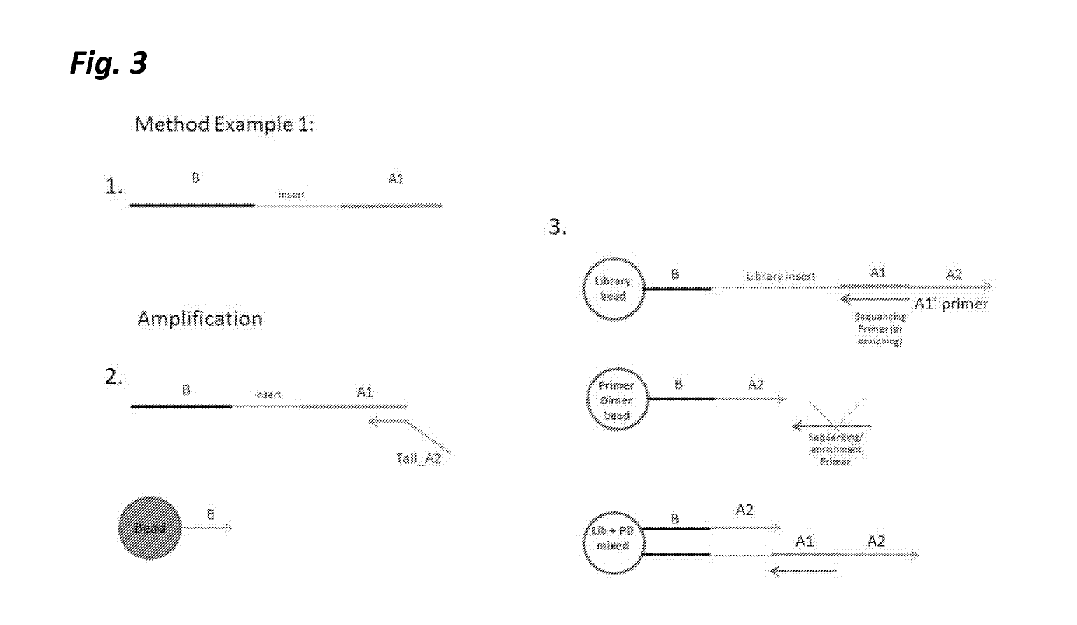

In some embodiments, provided is a method comprising: (a) providing a plurality of template polynucleotides which comprises a first adaptor sequence (B adaptor sequence), an insert template sequence, and a second adaptor sequence (A1 adaptor sequence); (b) forming a reaction mixture (FIG. 3 Amplification) by contacting: (i) the plurality of template polynucleotides, (ii) at least one bead having a plurality of a capture primer attached thereon, wherein the capture primer is capable of hybridizing to the first adaptor sequence (B adaptor sequence), (iii) a plurality of a solution-phase primer (Tail_A2), wherein the solution-phase primer comprises, in a 5′ to 3′ direction, a fourth adaptor sequence and a third adaptor sequence, wherein the third adaptor sequence is capable of hybridizing to the second adaptor sequence (A1) or the complement sequence of the second adaptor sequence; (c) subjecting the reaction mixture to a nucleic acid amplification condition, thereby generating a mixture of different types of templated beads, where each templated bead is attached with a plurality of polynucleotides (FIG. 3 Sequential Primer Sequencing), and where the different types of templated beads include (i) library beads, (ii) primer-dimer-beads, and (iii) mixed library-primer-dimer beads; and (d) sequencing (FIG. 3 Sequential Primer Sequencing) by contacting the mixture of different types of templated beads with a sequencing primer that is capable of hybridizing to the second adaptor sequence (A1).

In some embodiments, in step (c), the nucleic acid amplification condition generates a mixture of different types of templated beads, where the mixture includes (i) library beads, (ii) primer-dimer-beads, and (iii) mixed library-primer-dimer beads. The library beads include beads that are attached with a plurality of substantially monoclonal copies of a library molecule, where the library molecule includes the capture primer sequence (B), the insert sequence, the second adaptor sequence (A1), and the complement sequence of the fourth adaptor sequence (A2). The primer-dimer beads include beads that are attached with a plurality of primer-dimers, where the primer-dimers include the capture primer sequence (B) and the complement of the fourth adaptor sequence (A2). The mixed library-primer-dimer beads include a plurality of primer-dimers and library molecules attached to the same bead, where the primer-dimers include the capture primer sequence (B) and the complement sequence of the fourth adaptor sequence (A2), and where library molecules include the capture primer sequence (B), the insert sequence, the second adaptor sequence (A1), and the complement sequence of the fourth adaptor sequence (A2).

In some embodiments, in step (d) the sequencing primer hybridizes to the second adaptor sequence (A1) of the library beads and the mixed library-primer-dimer beads, but the sequencing primer exhibits substantially reduced hybridization to the primer-dimer beads because the primer-dimer beads lack a second adaptor sequence. This selective hybridization results in selectively sequencing the library beads and the mixed library-primer-dimer beads, and reduced sequencing of the primer-dimer beads. In some embodiments, the sequencing step (d) also includes contacting the mixture of different types of templated beads with a sequencing primer, a polymerase and a plurality of nucleotides, under conditions suitable for polymerase-catalyzed nucleotide incorporation.

In some embodiments, in step (d) the sequencing can be conducting on a high throughput sequencing platform. For example, the high throughput sequencing platform includes sequencing by oligonucleotide probe ligation and detection (e.g., SOLiD™), probe-anchor ligation sequencing (e.g., Complete Genomics or Polonator™), sequence-by-synthesis (e.g., Illumina), pyrophosphate sequencing (e.g., 454 Life Sciences), ion-sensitive sequencing (e.g., Personal Genome Machine (PGM™) and Ion Proton™ Sequencer, both from Ion Torrent Systems, Inc.) and single molecule sequencing platforms (e.g., Helicos™).

In some embodiments, step (d) can be preceded by an optional enrichment step, which comprises: contacting the different types of templated beads (FIG. 3 Sequential Primer Sequencing) with a plurality of enrichment primers that are capable of hybridizing to a sequence within the second adaptor sequence (A1) or are capable of hybridizing to the complement sequence of the second adaptor sequence, wherein the plurality of enrichment primers include an affinity moiety. The enrichment step generates pre-enrichment complexes which include non-complexed primer-dimer beads, and library beads hybridized to the enrichment primers and mixed library-primer-dimer beads hybridized to the enrichment primers. In some embodiments, the enrichment primers and the sequencing primers hybridize to the same adaptor sequence. In some embodiments, the affinity moiety binds a receptor moiety. In some embodiments, the affinity moiety comprises biotin and the receptor moiety comprises an avidin-like moiety. The optional enrichment step can be followed by an optional separating step, which comprises: separating the pre-enrichment complexes from the non-complexed primer-dimer beads, thereby enriching for templated beads that carry library molecules having the second adaptor sequence (A1) or the complement sequence of the second adaptor sequence. The separating step can be practiced by contacting the affinity moiety (on the enrichment primers) with a purification bead that is attached to one or more receptor moieties, to form the enrichment complex. In some embodiments, the enrichment complex comprises a purification bead (having a receptor moiety) bound to a library bead (having an affinity moiety) or a mixed library primer-dimer bead (having an affinity moiety). The enrichment complex is separated or removed from the plurality of non-complexed primer-dimer beads, thereby generating an enriched population of templated beads that carry library molecules having the second adaptor sequence (A1) or the complement sequence of the second adaptor sequence. In some embodiments, the purification bead comprises a paramagnetic bead that can be moved/manipulated with a magnet.

In some embodiments, in steps (b) and (c), the nucleic acid amplification condition comprises a primer extension reaction. In some embodiments, the nucleic acid amplification condition comprises an isothermal amplification condition or a thermocycling amplification condition (e.g., thermocycling PCR). In some embodiments, the reaction mixture is used to conduct nucleic acid amplification, and the reaction mixture comprises a polymerase and a plurality of nucleotides. In some embodiments, the reaction mixture comprises a recombinase. In some embodiments, the recombinase is uvsX. In some embodiments, the reaction mixture comprises at least one recombinase accessory protein. In some embodiments, the reaction mixture comprises at least one recombinase loading protein and/or at least one single-stranded binding protein. In some embodiments, at least one recombinase loading protein is uvsY. In some embodiments, at least one single-stranded binding protein is gp32. In some embodiments, any one or any combination of the uvsX, uvsY and/or gp32 can be from T4 bacteriophage.

In some embodiments, in step (b), the reaction mixture is contained in a water droplet in an oil-and-water emulsion which provides compartmentalization. In some embodiments, the reaction mixture is part of a single continuous liquid phase that does not provide compartmentalization. In some embodiments, the single continuous liquid phase lacks an oil and water emulsion. In some embodiments, the single continuous liquid phase is an aqueous phase.

In some embodiments, in step (d), the sequencing step includes: depositing to a reaction site on a support, at least one bead attached to polynucleotides, wherein the support contains plurality of reaction sites arranged in an array. In some embodiments, the beads that are deposited include primer-dimer beads, mixed library-primer-dimer beads, and library beads (FIG. 3). In some embodiments, at least one reaction site is operatively coupled to a sensor. In some embodiments, beads attached to different polynucleotide populations are deposited on different reaction sites on the same array. In some embodiments, the different polynucleotides are sequenced in parallel at the different reaction sites of the array. In some embodiments, the individual reaction sites in the array are operatively coupled to at least one sensor.

In some embodiments, in step (d), the sequencing step comprises sequencing the library molecules attached to the beads, and wherein the sequencing is conducted at the reaction site. In some embodiments, the sequencing comprises detecting at least one nucleotide incorporation byproduct using the at least one sensor operatively coupled to the reaction site. In some embodiments, at least one nucleotide incorporation byproduct is selected from hydrogen ions, hydroxyl ions, pyrophosphate, charge transfer, and heat. In some embodiments, the sensor detects a change in pH. In some embodiments, the sensor comprises a field effect transistor (FET). In some embodiments, the sensor comprises a chemical field effect transistor (chemFET). In some embodiments, the sensor comprises an ion-sensitive field effect transistor (ISFET).

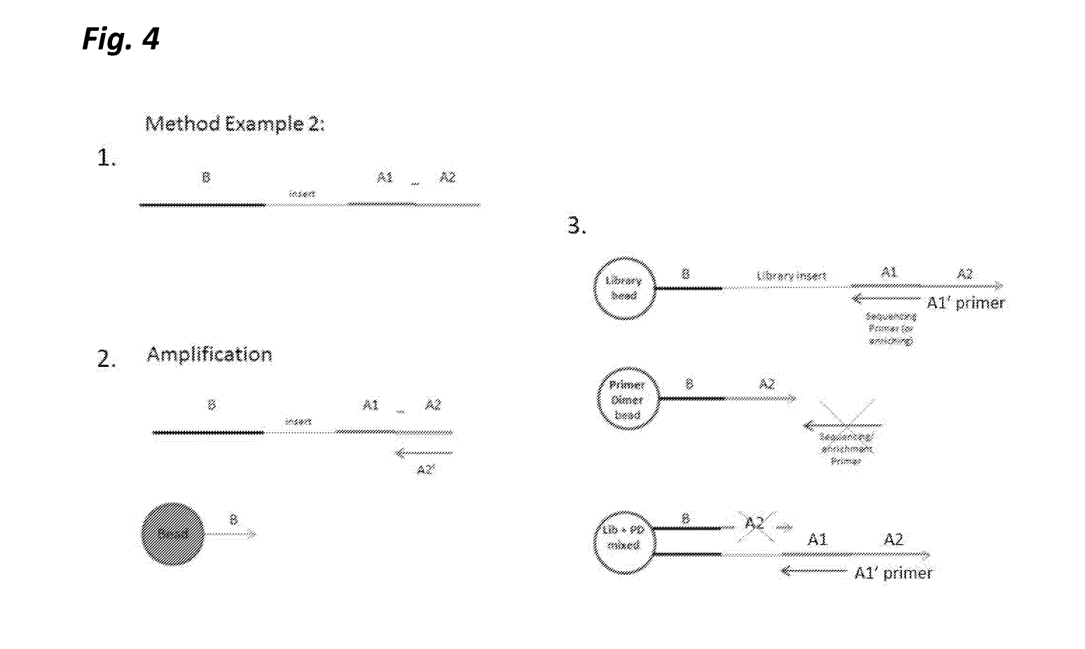

In some embodiments, provided is a method comprising: (a) providing a plurality of template polynucleotides which comprises a first adaptor sequence (B adaptor sequence), an insert template sequence, a second adaptor sequence (A1 adaptor sequence), and a third adaptor sequence (A2 adaptor sequence); (b) forming a reaction mixture (FIG. 4 Amplification) by contacting: (i) the plurality of template polynucleotides, (ii) at least one bead having a plurality of a capture primer attached thereon, wherein the capture primer is capable of hybridizing to the first adaptor sequence (B adaptor sequence), (iii) a plurality of a solution-phase primer (A2′), wherein the solution-phase primer comprises a sequence that is capable of hybridizing to the third adaptor sequence (A2) or the complement sequence of the second adaptor sequence; (c) subjecting the reaction mixture to a nucleic acid amplification condition, thereby generating a mixture of different types of templated beads, where each templated bead is attached with a plurality of polynucleotides (FIG. 4 Sequential Primer Sequencing), where the different types of templated beads include (i) library beads, (ii) primer-dimer-beads, and (iii) mixed library-primer-dimer beads; and (d) sequencing (FIG. 4 Sequential Primer Sequencing) by contacting the mixture of different types of templated beads with a sequencing primer that is capable of hybridizing to the complement of the second adaptor sequence (A1).

In some embodiments, in step (b), the solution-phase primer (A2′) is a non-tailed primer.

In some embodiments, in step (c), the nucleic acid amplification condition generates a mixture of different types of templated beads which includes (i) library beads, (ii) primer-dimer-beads, and (iii) mixed library-primer-dimer beads. The library beads include beads that are attached with a plurality of substantially monoclonal copies of a library molecule, where the library molecule includes the capture primer sequence (B), the library insert sequence, the second adaptor sequence (A1), and the third adaptor sequence (A2). The primer-dimer beads include beads that are attached with a plurality of primer-dimers, where the primer-dimers include the capture primer sequence (B) and the third adaptor sequence (A2). The mixed library-primer-dimer beads include primer-dimers and library molecules attached to the same bead, where the primer-dimers include the capture primer sequence (B) and the third adaptor sequence (A2), and where library molecules include the capture primer sequence (B), the library insert sequence, the second adaptor sequence (A1), and the third adaptor sequence (A2).

In some embodiments, in step (d) the sequencing primer hybridizes to the second adaptor sequence (A1) of the library beads and the mixed library-primer-dimer beads, but the sequencing primer exhibits substantially reduced hybridization to the primer-dimer beads. This selective hybridization results in selectively sequencing the library beads and the mixed library-primer-dimer beads, and reduced sequencing of the primer-dimer beads. In some embodiments, the sequencing step (d) also includes contacting the mixture of different types of templated beads with a sequencing primer, a polymerase and a plurality of nucleotides, under conditions suitable for polymerase-catalyzed nucleotide incorporation.

In some embodiments, in step (d) the sequencing can be conducting on a high throughput sequencing platform. For example, the high throughput sequencing platform includes sequencing by oligonucleotide probe ligation and detection (e.g., SOLiD™), probe-anchor ligation sequencing (e.g., Complete Genomics or Polonator™), sequence-by-synthesis (e.g., Illumina), pyrophosphate sequencing (e.g., 454 Life Sciences), ion-sensitive sequencing (e.g., Personal Genome Machine (PGM™) and Ion Proton™ Sequencer, both from Ion Torrent Systems, Inc.) and single molecule sequencing platforms (e.g., Helicos™).

In some embodiments, step (d) can be preceded by an optional enrichment step, which comprises: contacting the different types of templated beads (FIG. 4 Sequential Primer Sequencing) with a plurality of enrichment primers that are capable of hybridizing to a sequence within the second adaptor sequence (A1) or are capable of hybridizing to the complement sequence of the second adaptor sequence, wherein the plurality of enrichment primers include an affinity moiety. The enrichment step generates pre-enrichment complexes which include non-complexed primer-dimer beads, and library beads hybridized to the enrichment primers and mixed library-primer-dimer beads hybridized to the enrichment primers. In some embodiments, the enrichment primers and the sequencing primers hybridize to the same adaptor sequence. In some embodiments, the affinity moiety binds a receptor moiety. In some embodiments, the affinity moiety comprises biotin and the receptor moiety comprises an avidin-like moiety. The optional enrichment step can be followed by an optional separating step, which comprises: separating the pre-enrichment complexes from the non-complexed primer-dimer beads, thereby enriching for templated beads that carry library molecules having the second adaptor sequence (A1) or the complement sequence of the second adaptor sequence. The separating step can be practiced by contacting the affinity moiety (on the enrichment primers) with a purification bead that is attached to one or more receptor moieties, to form the enrichment complex. In some embodiments, the enrichment complex comprises a purification bead (having a receptor moiety) bound to a library bead (having an affinity moiety) or a mixed library primer-dimer bead (having an affinity moiety). The enrichment complex is separated or removed from the plurality of non-complexed primer-dimer beads, thereby generating an enriched population of templated beads that carry library molecules having the second adaptor sequence (A1) or the complement sequence of the second adaptor sequence. In some embodiments, the purification bead comprises a paramagnetic bead that can be moved/manipulated with a magnet.

In some embodiments, in steps (b) and (c), the nucleic acid amplification condition comprises a primer extension reaction. In some embodiments, the nucleic acid amplification condition comprises an isothermal amplification condition or a thermocycling amplification condition (e.g., thermocycling PCR). In some embodiments, the reaction mixture is used to conduct nucleic acid amplification, and the reaction mixture comprises a polymerase and a plurality of nucleotides. In some embodiments, the reaction mixture comprises a recombinase. In some embodiments, the recombinase is uvsX. In some embodiments, the reaction mixture comprises at least one recombinase accessory protein. In some embodiments, the reaction mixture comprises at least one recombinase loading protein and/or at least one single-stranded binding protein. In some embodiments, at least one recombinase loading protein is uvsY. In some embodiments, at least one single-stranded binding protein is gp32. In some embodiments, any one or any combination of the uvsX, uvsY and/or gp32 can be from T4 bacteriophage.

In some embodiments, in step (b), the reaction mixture is contained in a water droplet in an oil-and-water emulsion which provides compartmentalization. In some embodiments, the reaction mixture is part of a single continuous liquid phase that does not provide compartmentalization. In some embodiments, the single continuous liquid phase lacks an oil and water emulsion. In some embodiments, the single continuous liquid phase is an aqueous phase.

In some embodiments, in step (d), the sequencing step includes: depositing to a reaction site on a support, at least one bead attached to polynucleotides, wherein the support contains plurality of reaction sites arranged in an array. In some embodiments, the beads that are deposited include primer-dimer beads, mixed library-primer-dimer beads, and library beads (FIG. 4). In some embodiments, at least one reaction site is operatively coupled to a sensor. In some embodiments, beads attached to different polynucleotide populations are deposited on different reaction sites on the same array. In some embodiments, the different polynucleotides are sequenced in parallel at the different reaction sites of the array. In some embodiments, the individual reaction sites in the array are operatively coupled to at least one sensor.

In some embodiments, in step (d), the sequencing step comprises sequencing the library molecules attached to the beads, and wherein the sequencing is conducted at the reaction site. In some embodiments, the sequencing comprises detecting at least one nucleotide incorporation byproduct using the at least one sensor operatively coupled to the reaction site. In some embodiments, at least one nucleotide incorporation byproduct is selected from hydrogen ions, hydroxyl ions, pyrophosphate, charge transfer, and heat. In some embodiments, the sensor detects a change in pH. In some embodiments, the sensor comprises a field effect transistor (FET). In some embodiments, the sensor comprises a chemical field effect transistor (chemFET). In some embodiments, the sensor comprises an ion-sensitive field effect transistor (ISFET).

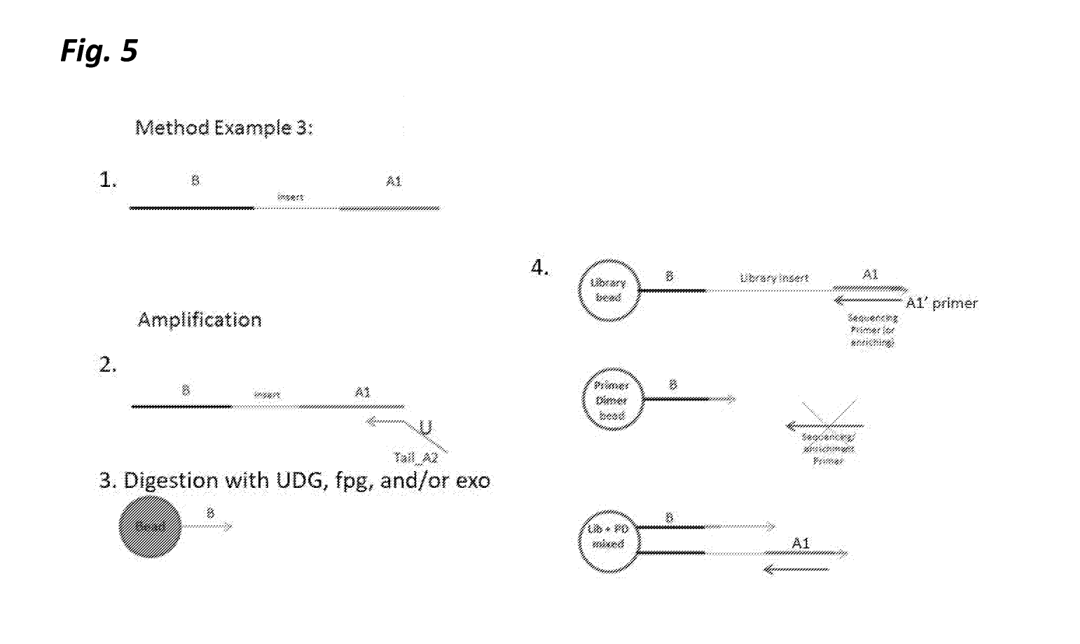

In some embodiments, provided is a method comprising: (a) providing a plurality of template polynucleotides which comprises a first adaptor sequence (B adaptor sequence), an insert template sequence, and a second adaptor sequence (A1 adaptor sequence); (b) forming a reaction mixture (FIG. 5 Amplification) by contacting: (i) the plurality of template polynucleotides, (ii) at least one bead having a plurality of a capture primer attached thereon, wherein the capture primer is capable of hybridizing to the first adaptor sequence (B adaptor sequence), (iii) a plurality of a solution-phase primer (Tail_A2), wherein the solution-phase primer comprises, in a 5′ to 3′ direction, a fourth adaptor sequence and a third adaptor sequence, wherein the fourth adaptor sequence includes at least one nucleotide having a uracil base, and wherein the third adaptor sequence is capable of hybridizing to the second adaptor sequence (A1) or the complement sequence of the second adaptor sequence; (c) subjecting the reaction mixture to a nucleic acid amplification condition, thereby generating a mixture of different types of templated beads, where each templated bead is attached with a plurality of polynucleotides that contain at least one primer-derived uracil base, and where the different types of templated beads include (i) library beads, (ii) primer-dimer-beads, and (iii) mixed library-primer-dimer beads; and (d) cleaving the uracil bases, by contacting the mixture of different types of templated beads with a cleaving agent that comprises uracil DNA glycosylase (UDG), formamidopyrimidine DNA glycosylase (Fpg) and/or an appropriate exonuclease (FIG. 5 Digestion with UDG, fpg, and/or exo), to generate templated beads attached with a plurality of polynucleotides that contain a truncated third adaptor sequence; and (e) sequencing (FIG. 5 Sequential Primer Sequencing) by contacting the mixture of different types of templated beads attached with a plurality of polynucleotides that contain a truncated third adaptor sequence with a sequencing primer that is capable of hybridizing to the second adaptor sequence (A1).

In some embodiments, in step (c), the nucleic acid amplification condition generates a mixture of different types of templated beads, where the mixture includes (i) library beads, (ii) primer-dimer-beads, and (iii) mixed library-primer-dimer beads. The library beads include beads that are attached with a plurality of substantially monoclonal copies of a library molecule, where the library molecule includes the capture primer sequence (B), the insert sequence, the second adaptor sequence (A1), and the truncated complement sequence of the fourth adaptor sequence (A2). The primer-dimer beads include beads that are attached with a plurality of primer-dimers, where the primer-dimers include the capture primer sequence (B) and the truncated complement of the fourth adaptor sequence (A2). The mixed library-primer-dimer beads include a plurality of primer-dimers and library molecules attached to the same bead, where the primer-dimers include the capture primer sequence (B) and the truncated complement sequence of the fourth adaptor sequence (A2), and where library molecules include the capture primer sequence (B), the insert sequence, the second adaptor sequence (A1), and the complement sequence of the fourth adaptor sequence (A2).

In some embodiments, in step (d), the cleaving agent can also include a DNA polymerase, such as PolI, T4 PNK or Klenow. The cleaving agent can optionally include an antibody that inhibits the activity of the DNA polymerase and 3′-5′ exonuclease activities at ambient temperatures.

In some embodiments, in step (d), the mixture of different types of templated beads includes (i) library beads, (ii) primer-dimer-beads, and (iii) mixed library-primer-dimer beads, which comprise beads attached with a plurality of polynucleotides that contain a primer-derived third adaptor sequence that can be cleaved by the cleaving agent to generate templated beads attached with a plurality of polynucleotides that contain a truncated third adaptor sequence.

In some embodiments, in step (e) the sequencing primer hybridizes to the second adaptor sequence (A1) of the library beads and the mixed library-primer-dimer beads, but the sequencing primer exhibits substantially reduced hybridization to the primer-dimer beads because the primer-dimer beads lack a second adaptor sequence. Additionally, the truncated third adaptor sequence on the different types of templated beads will reduce the possibility of the hybridization between the sequencing primer and the third adaptor sequence. This selective hybridization results in selectively sequencing the library beads and the mixed library-primer-dimer beads, and reduced sequencing of the primer-dimer beads. In some embodiments, the sequencing step (e) also includes contacting the mixture of different types of templated beads with a sequencing primer, a polymerase and a plurality of nucleotides, under conditions suitable for polymerase-catalyzed nucleotide incorporation.

In some embodiments, in step (e) the sequencing can be conducting on a high throughput sequencing platform. For example, the high throughput sequencing platform includes sequencing by oligonucleotide probe ligation and detection (e.g., SOLiD™), probe-anchor ligation sequencing (e.g., Complete Genomics or Polonator™), sequence-by-synthesis (e.g., Illumina), pyrophosphate sequencing (e.g., 454 Life Sciences), ion-sensitive sequencing (e.g., Personal Genome Machine (PGM™) and Ion Proton™ Sequencer, both from Ion Torrent Systems, Inc.) and single molecule sequencing platforms (e.g., Helicos™).

In some embodiments, step (d) or (e) can be preceded by an optional enrichment step, which comprises: contacting the different types of templated beads (FIG. 5 Sequential Primer Sequencing) with a plurality of enrichment primers that are capable of hybridizing to a sequence within the second adaptor sequence (A1) or are capable of hybridizing to the complement sequence of the second adaptor sequence, wherein the plurality of enrichment primers include an affinity moiety. The enrichment step generates pre-enrichment complexes which include non-complexed primer-dimer beads, and library beads hybridized to the enrichment primers and mixed library-primer-dimer beads hybridized to the enrichment primers. In some embodiments, the enrichment primers and the sequencing primers hybridize to the same adaptor sequence. In some embodiments, the affinity moiety binds a receptor moiety. In some embodiments, the affinity moiety comprises biotin and the receptor moiety comprises an avidin-like moiety. The optional enrichment step can be followed by an optional separating step, which comprises: separating the pre-enrichment complexes from the non-complexed primer-dimer beads, thereby enriching for templated beads that carry library molecules having the second adaptor sequence (A1) or the complement sequence of the second adaptor sequence. The separating step can be practiced by contacting the affinity moiety (on the enrichment primers) with a purification bead that is attached to one or more receptor moieties, to form the enrichment complex. In some embodiments, the enrichment complex comprises a purification bead (having a receptor moiety) bound to a library bead (having an affinity moiety) or a mixed library primer-dimer bead (having an affinity moiety). The enrichment complex is separated or removed from the plurality of non-complexed primer-dimer beads, thereby generating an enriched population of templated beads that carry library molecules having the second adaptor sequence (A1) or the complement sequence of the second adaptor sequence. In some embodiments, the purification bead comprises a paramagnetic bead that can be moved/manipulated with a magnet.

In some embodiments, in steps (b) and (c), the nucleic acid amplification condition comprises a primer extension reaction. In some embodiments, the nucleic acid amplification condition comprises an isothermal amplification condition or a thermocycling amplification condition (e.g., thermocycling PCR). In some embodiments, the reaction mixture is used to conduct nucleic acid amplification, and the reaction mixture comprises a polymerase and a plurality of nucleotides. In some embodiments, the reaction mixture comprises a recombinase. In some embodiments, the recombinase is uvsX. In some embodiments, the reaction mixture comprises at least one recombinase accessory protein. In some embodiments, the reaction mixture comprises at least one recombinase loading protein and/or at least one single-stranded binding protein. In some embodiments, at least one recombinase loading protein is uvsY. In some embodiments, at least one single-stranded binding protein is gp32. In some embodiments, any one or any combination of the uvsX, uvsY and/or gp32 can be from T4 bacteriophage.

In some embodiments, in step (b), the reaction mixture is contained in a water droplet in an oil-and-water emulsion which provides compartmentalization. In some embodiments, the reaction mixture is part of a single continuous liquid phase that does not provide compartmentalization. In some embodiments, the single continuous liquid phase lacks an oil and water emulsion. In some embodiments, the single continuous liquid phase is an aqueous phase.

In some embodiments, in step (e), the sequencing method includes: depositing to a reaction site on a support, at least one bead attached to polynucleotides, wherein the support contains plurality of reaction sites arranged in an array. In some embodiments, the beads that are deposited include primer-dimer beads, mixed library-primer-dimer beads, and library beads (FIG. 5). In some embodiments, at least one reaction site is operatively coupled to a sensor. In some embodiments, beads attached to different polynucleotide populations are deposited on different reaction sites on the same array. In some embodiments, the different polynucleotides are sequenced in parallel at the different reaction sites of the array. In some embodiments, the individual reaction sites in the array are operatively coupled to at least one sensor.

In some embodiments, in step (e), the sequencing method comprises sequencing the library molecules attached to the beads, and wherein the sequencing is conducted at the reaction site. In some embodiments, the sequencing comprises detecting at least one nucleotide incorporation byproduct using the at least one sensor operatively coupled to the reaction site. In some embodiments, at least one nucleotide incorporation byproduct is selected from hydrogen ions, hydroxyl ions, pyrophosphate, charge transfer, and heat. In some embodiments, the sensor detects a change in pH. In some embodiments, the sensor comprises a field effect transistor (FET). In some embodiments, the sensor comprises a chemical field effect transistor (chemFET). In some embodiments, the sensor comprises an ion-sensitive field effect transistor (ISFET).

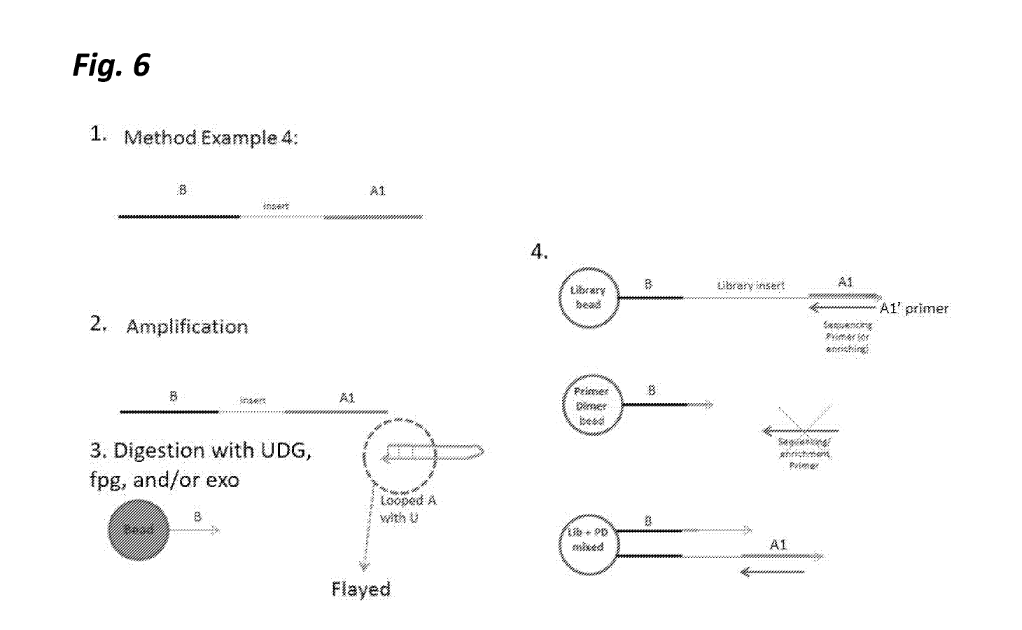

In some embodiments, provided is a method comprising: (a) providing a plurality of template polynucleotides which comprises a first adaptor sequence (B adaptor sequence), an insert template sequence, and a second adaptor sequence (A1 adaptor sequence); (b) forming a first reaction mixture (FIG. 6 Amplification) by contacting: (i) the plurality of template polynucleotides, and (ii) a plurality of a solution-phase hairpin primer (FIG. 6 Flayed), wherein the solution-phase hairpin primer includes self-complementary A2 adaptor sequences at its 5′ and 3′ ends that bracket a non-complementary region so that that solution-phase hairpin primers can form a hairpin or stem-loop structure, wherein the solution-phase hairpin primers comprise at least one uracil base at any position, and wherein the solution-phase hairpin primers include a portion in its 3′ region (and optionally includes a portion in its loop region) a section that can hybridize with a sequence near the terminal end of the second adaptor sequence (A1) of the template polynucleotide; (c) subjecting the first reaction mixture to a first nucleic acid amplification condition, thereby generating a plurality of hairpin amplicon duplexes which include two strands, wherein (i) the first strand includes (5′ to 3′) the capture primer sequence (B), the insert sequence and the second adaptor sequence (A1), wherein (ii) the second strand includes (5′ to 3′) the hairpin adaptor containing at least one uracil, the complementary sequence of the second adaptor sequence, a complementary sequence of the insert sequence, and a complementary sequence of the capture primer sequence; (d) cleaving the uracil bases, by contacting the hairpin amplicon duplexes with a cleaving agent that comprises uracil DNA glycosylase (UDG), formamidopyrimidine DNA glycosylase (Fpg) and/or an appropriate exonuclease (FIG. 6 Digestion with UDG, fpg, and/or exo), to generate cleaved duplexes having two strands, wherein the first strand includes (5′ to 3′) the capture primer sequence (B), the insert sequence, the second adaptor sequence (A1) and a truncated A2 adaptor sequence, wherein the second strand includes (5′ to 3′) a truncated complement of the A2 adaptor sequence, the complementary sequence of the second adaptor sequence, a complementary sequence of the insert sequence, and a complementary sequence of the capture primer sequence; (e) forming a second reaction mixture by contacting: (i) the cleaved duplexes, (ii) at least one bead having a plurality of a capture primer attached thereon, wherein the capture primer is capable of hybridizing to the first adaptor sequence (B adaptor sequence), and (iii) a plurality of a solution-phase primer (Tail_A2), wherein the solution-phase primer comprises, in a 5′ to 3′ direction, a truncated fourth adaptor sequence (trun-A2) and a third adaptor sequence which is capable of hybridizing to the second adaptor sequence (A1) or the complement sequence of the second adaptor sequence; (f) subjecting the second reaction mixture to a second nucleic acid amplification condition, thereby generating a mixture of different types of templated beads, where each templated bead is attached with a plurality of polynucleotides that contain at least one primer-derived uracil base, and where the different types of templated beads include (i) library beads, (ii) primer-dimer-beads, and (iii) mixed library-primer-dimer beads; and (g) sequencing (FIG. 6 Sequential Primer Sequencing) by contacting the mixture of different types of templated beads attached with a plurality of polynucleotides that contain a truncated third adaptor sequence with a sequencing primer that is capable of hybridizing to the second adaptor sequence (A1).

In some embodiments, in step (d), the cleaving agent can also include a DNA polymerase, such as PolI, T4 PNK or Klenow. The cleaving agent can optionally include an antibody that inhibits the activity of the DNA polymerase and 3′-5′ exonuclease activities at ambient temperatures.

In some embodiments, in step (d), the mixture of different types of templated beads includes (i) library beads, (ii) primer-dimer-beads, and (iii) mixed library-primer-dimer beads, which comprise beads attached with a plurality of polynucleotides that contain a primer-derived third adaptor sequence that can be cleaved by the cleaving agent to generate templated beads attached with a plurality of polynucleotides that contain a truncated third adaptor sequence.

In some embodiments, in step (e), the second nucleic acid amplification condition generates a mixture of different types of templated beads, where the mixture includes (i) library beads, (ii) primer-dimer-beads, and (iii) mixed library-primer-dimer beads. The library beads include beads that are attached with a plurality of substantially monoclonal copies of a library molecule, where the library molecule includes the capture primer sequence (B), the insert sequence, the second adaptor sequence (A1), and the truncated complement sequence of the fourth adaptor sequence (A2). The primer-dimer beads include beads that are attached with a plurality of primer-dimers, where the primer-dimers include the capture primer sequence (B) and the truncated complement of the fourth adaptor sequence (A2). The mixed library-primer-dimer beads include a plurality of primer-dimers and library molecules attached to the same bead, where the primer-dimers include the capture primer sequence (B) and the truncated complement sequence of the fourth adaptor sequence (A2), and where library molecules include the capture primer sequence (B), the insert sequence, the second adaptor sequence (A1), and the complement sequence of the fourth adaptor sequence (A2).

In some embodiments, in step (g) the sequencing primer hybridizes to the second adaptor sequence (A1) of the library beads and the mixed library-primer-dimer beads, but the sequencing primer exhibits substantially reduced hybridization to the primer-dimer beads because the primer-dimer beads lack a second adaptor sequence. Additionally, the truncated third adaptor sequence on the different types of templated beads will reduce the possibility of the hybridization between the sequencing primer and the third adaptor sequence. This selective hybridization results in selectively sequencing the library beads and the mixed library-primer-dimer beads, and reduced sequencing of the primer-dimer beads. In some embodiments, the sequencing step (e) also includes contacting the mixture of different types of templated beads with a sequencing primer, a polymerase and a plurality of nucleotides, under conditions suitable for polymerase-catalyzed nucleotide incorporation.

In some embodiments, in step (g) the sequencing can be conducting on a high throughput sequencing platform. For example, the high throughput sequencing platform includes sequencing by oligonucleotide probe ligation and detection (e.g., SOLiD™), probe-anchor ligation sequencing (e.g., Complete Genomics or Polonator™), sequence-by-synthesis (e.g., Illumina), pyrophosphate sequencing (e.g., 454 Life Sciences), ion-sensitive sequencing (e.g., Personal Genome Machine (PGM™) and Ion Proton™ Sequencer, both from Ion Torrent Systems, Inc.) and single molecule sequencing platforms (e.g., Helicos™).

In some embodiments, step (e), (f) or (g) can be preceded by an optional enrichment step, which comprises: contacting the different types of templated beads (FIG. 6 Sequential Primer Sequencing) with a plurality of enrichment primers that are capable of hybridizing to a sequence within the second adaptor sequence (A1) or are capable of hybridizing to the complement sequence of the second adaptor sequence, wherein the plurality of enrichment primers include an affinity moiety. The enrichment step generates pre-enrichment complexes which include non-complexed primer-dimer beads, and library beads hybridized to the enrichment primers and mixed library-primer-dimer beads hybridized to the enrichment primers. In some embodiments, the enrichment primers and the sequencing primers hybridize to the same adaptor sequence. In some embodiments, the affinity moiety binds a receptor moiety. In some embodiments, the affinity moiety comprises biotin and the receptor moiety comprises an avidin-like moiety. The optional enrichment step can be followed by an optional separating step, which comprises: separating the pre-enrichment complexes from the non-complexed primer-dimer beads, thereby enriching for templated beads that carry library molecules having the second adaptor sequence (A1) or the complement sequence of the second adaptor sequence. The separating step can be practiced by contacting the affinity moiety (on the enrichment primers) with a purification bead that is attached to one or more receptor moieties, to form the enrichment complex. In some embodiments, the enrichment complex comprises a purification bead (having a receptor moiety) bound to a library bead (having an affinity moiety) or a mixed library primer-dimer bead (having an affinity moiety). The enrichment complex is separated or removed from the plurality of non-complexed primer-dimer beads, thereby generating an enriched population of templated beads that carry library molecules having the second adaptor sequence (A1) or the complement sequence of the second adaptor sequence. In some embodiments, the purification bead comprises a paramagnetic bead that can be moved/manipulated with a magnet.

In some embodiments, in steps (b) and (c), the nucleic acid amplification condition comprises a primer extension reaction. In some embodiments, the nucleic acid amplification condition comprises an isothermal amplification condition or a thermocycling amplification condition (e.g., thermocycling PCR). In some embodiments, the reaction mixture is used to conduct nucleic acid amplification, and the reaction mixture comprises a polymerase and a plurality of nucleotides. In some embodiments, the reaction mixture comprises a recombinase. In some embodiments, the recombinase is uvsX. In some embodiments, the reaction mixture comprises at least one recombinase accessory protein. In some embodiments, the reaction mixture comprises at least one recombinase loading protein and/or at least one single-stranded binding protein. In some embodiments, at least one recombinase loading protein is uvsY. In some embodiments, at least one single-stranded binding protein is gp32. In some embodiments, any one or any combination of the uvsX, uvsY and/or gp32 can be from T4 bacteriophage.

In some embodiments, in step (b), the reaction mixture is contained in a water droplet in an oil-and-water emulsion which provides compartmentalization. In some embodiments, the reaction mixture is part of a single continuous liquid phase that does not provide compartmentalization. In some embodiments, the single continuous liquid phase lacks an oil and water emulsion. In some embodiments, the single continuous liquid phase is an aqueous phase.

In some embodiments, in step (g), the sequencing step includes: depositing to a reaction site on a support, at least one bead attached to polynucleotides, wherein the support contains plurality of reaction sites arranged in an array. In some embodiments, the beads that are deposited include primer-dimer beads, mixed library-primer-dimer beads, and library beads (FIG. 6). In some embodiments, at least one reaction site is operatively coupled to a sensor. In some embodiments, beads attached to different polynucleotide populations are deposited on different reaction sites on the same array. In some embodiments, the different polynucleotides are sequenced in parallel at the different reaction sites of the array. In some embodiments, the individual reaction sites in the array are operatively coupled to at least one sensor.

In some embodiments, in step (g), the sequencing step comprises sequencing the library molecules attached to the beads, and wherein the sequencing is conducted at the reaction site. In some embodiments, the sequencing comprises detecting at least one nucleotide incorporation byproduct using the at least one sensor operatively coupled to the reaction site. In some embodiments, at least one nucleotide incorporation byproduct is selected from hydrogen ions, hydroxyl ions, pyrophosphate, charge transfer, and heat. In some embodiments, the sensor detects a change in pH. In some embodiments, the sensor comprises a field effect transistor (FET). In some embodiments, the sensor comprises a chemical field effect transistor (chemFET). In some embodiments, the sensor comprises an ion-sensitive field effect transistor (ISFET).

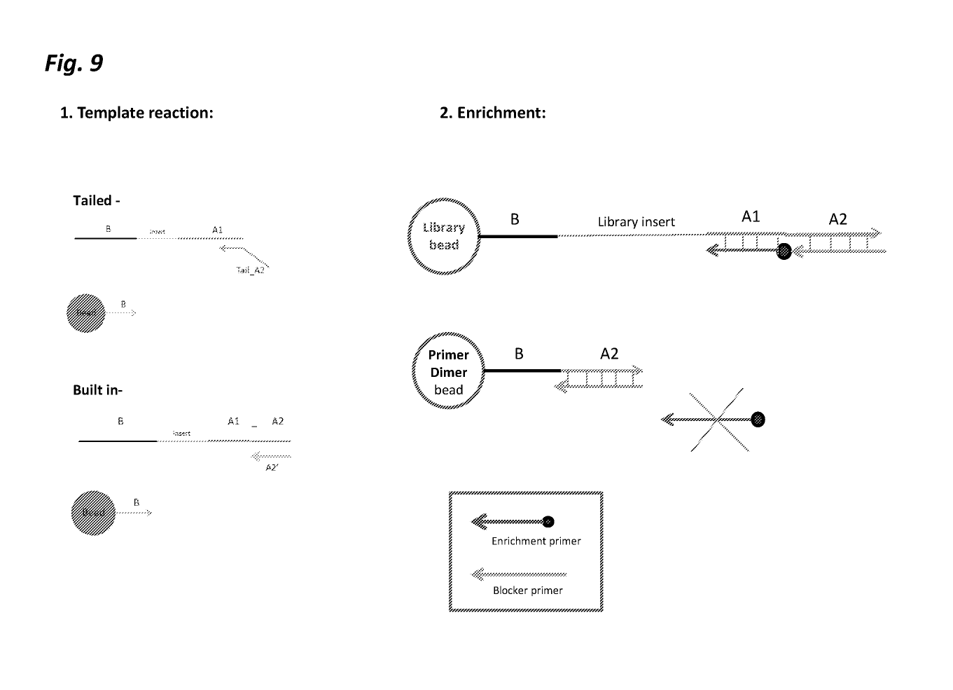

In some embodiments, provided are methods, as well as related systems, compositions, kits, and apparatuses for enriching template polynucleotides (FIG. 9), comprising: (a) providing a mixture of polynucleotides which include (i) a plurality of template polynucleotides and (ii) a plurality of primer dimer byproducts, wherein individual template polynucleotides from the plurality of template polynucleotides include, in a 5′ to 3′direction, a first universal adaptor sequence, a template sequence, a second universal adaptor sequence, and a third universal adaptor sequence, wherein individual primer dimer byproducts from the plurality of primer dimer byproducts include, in a 5′ to 3′ direction, a first universal adaptor sequence and a third universal adaptor sequence; (b) forming a plurality of pre-enrichment complexes by contacting the mixture of polynucleotides with a plurality of enrichment primers that are capable of hybridizing to at least a portion of the second universal adaptor sequence, wherein the enrichment primer includes an affinity moiety; (c) forming a plurality of blocked primer dimer complexes by contacting the mixture of polynucleotides with a plurality of blocking primers that are capable of hybridizing to at least a portion of the third universal adaptor sequence on the primer dimer byproducts; and (d) separating the plurality of pre-enrichment complexes from the plurality of blocked primer dimer complexes, thereby enriching the template polynucleotides.

In some embodiments, in step (a), the first, second and third universal adaptor sequences have different nucleotide sequences.

In some embodiments, in step (a), the second universal adaptor sequence is identical to, or is capable of, hybridizing to a sequencing primer.

In some embodiments, in step (a), at least one of the template polynucleotides is a soluble polynucleotide molecule or at least one of the template polynucleotides is attached to a support. In a particular embodiment at least one of the template polynucleotides is attached to a support as shown in FIG. 9. The support can be a planar support or a bead. In some embodiments, the 5′ end of the at least one template polynucleotide is attached to the support. For example, the template polynucleotide includes the first universal adaptor sequence which is attached directly or indirectly to the support. A plurality of substantially monoclonal copies of the template polynucleotide can be attached to a support (e.g., bead).

In some embodiments, in step (a), the mixture of polynucleotides includes (i) a plurality of template polynucleotides attached to a support and (ii) a plurality of primer dimer byproducts attached to a different support, wherein individual template polynucleotides from the plurality of template polynucleotides include, in a 5′ to 3′ direction, a first universal adaptor sequence, a template sequence, a second universal adaptor sequence, and a third universal adaptor sequence, wherein the 5′ or 3′ end of the individual template polynucleotides are attached to the support.

In some embodiments, in step (a), the support can be attached with a plurality of substantially monoclonal copies of the template polynucleotide.

In some embodiments, in step (a), the primer dimer byproducts are soluble nucleic acid molecules.

In some embodiments, steps (b) and (c) are performed sequentially in any order, or simultaneously.

In some embodiments, in steps (b) and (c), the enrichment primer and blocking primer can hybridize to the same template polynucleotides at the same time. For example, the enrichment primer hybridizes to the second universal sequence and the blocking primer hybridizes to the third universal adaptor sequence (see, e.g., FIG. 9). In some embodiments, the enrichment primer and the blocking primer hybridize to overlapping regions on the template polynucleotide, or hybridize to non-overlapping regions on the template polynucleotide.

In some embodiments, in step (b), the plurality of enrichment primers hybridize selectively to the second universal adaptor sequence. In some embodiments, the plurality of enrichment primers exhibit reduced hybridization to the first and third universal adaptor sequences.

In some embodiments, in step (b), the enrichment primer includes an affinity moiety which selectively binds a receptor moiety. In particular embodiments, the affinity moiety comprises biotin, and the receptor moiety comprises an avidin-like moiety.

In some embodiments, in step (c), the plurality of blocking primers hybridize selectively to the third universal adaptor sequence. In some embodiments, the plurality of blocking primers hybridize less selectively to the first and second universal adaptor sequences.

In some embodiments, in step (c), when the blocking primer hybridizes to the third universal adaptor sequence of the primer dimer byproduct, the enrichment primer is blocked from hybridizing to the third universal adaptor sequence of the same primer dimer byproduct.

In some embodiments, in step (c), the 3′ end of the blocking primer is not extendible in a primer extension reaction. For example, in a particular embodiment the 3′end of the blocking primer comprises a hydrogen or a moiety that blocks primer extension.

In some embodiments, in step (d), the blocked primer dimer complexes are not enriched with the pre-enrichment complexes.

In some embodiments, in step (d), the plurality of pre-enrichment complexes can be separated from the plurality of blocked primer dimer complexes by contacting the affinity moiety (on the enrichment primers) with a purification bead that is attached to one or more receptor moieties, to form an enrichment complex. In certain embodiments an enrichment complex comprises: a template polynucleotide bound to an enrichment primer and a blocking primer, and the enrichment primer (having an affinity moiety) is bound to a purification bead (having a receptor moiety). In particular embodiments an enrichment complex is separated or removed from the plurality of blocked primer dimer complexes, thereby generating an enriched population of template polynucleotides. In some embodiments, the purification bead comprises a paramagnetic bead that can be moved/manipulated with a magnet.

In some embodiments, the method further comprises step (e1): optionally discarding the plurality of blocked primer dimer complexes.

In some embodiments, the method further comprises step (e2): optionally amplifying the plurality of template polynucleotides from the plurality of enrichment complexes.

In some embodiments, the method further comprises step (f): sequencing the plurality of template polynucleotides from the plurality of enrichment complexes. The sequencing can be conducting on a high throughput sequencing platform. For example, the high throughput sequencing platform includes sequencing by oligonucleotide probe ligation and detection (e.g., SOLiD™), probe-anchor ligation sequencing (e.g., Complete Genomics or Polonator™), sequence-by-synthesis (e.g., Illumina), pyrophosphate sequencing (e.g., 454 Life Sciences), ion-sensitive sequencing (e.g., Personal Genome Machine (PGM™) and Ion Proton™ Sequencer, both from Ion Torrent Systems, Inc.) and single molecule sequencing platforms (e.g., Helicos™).

In some embodiments, in step (f), the sequencing step includes: depositing onto a reaction site on a sequencing support (e.g., planar support), at least one bead attached to polynucleotides, wherein the sequencing support contains plurality of reaction sites arranged in an array. In some embodiments, the beads that are deposited include primer-dimer beads, mixed library-primer-dimer beads, and library beads (FIG. 9). In some embodiments, at least one reaction site is operatively coupled to a sensor. In some embodiments, beads attached to different polynucleotide populations are deposited on different reaction sites on the same array. In some embodiments, the different polynucleotides are sequenced in parallel at the different reaction sites of the array. In some embodiments, the individual reaction sites in the array are operatively coupled to at least one sensor.

In some embodiments, in step (f), the sequencing step comprises sequencing the library molecules attached to the beads, and wherein the sequencing is conducted at the reaction site. In some embodiments, the sequencing comprises detecting at least one nucleotide incorporation byproduct using the at least one sensor which is operatively coupled to the reaction site. In some embodiments, the sensor detects the presence, or a change in concentration, of one or more nucleotide incorporation byproduct. In some embodiments, the nucleotide incorporation byproduct is selected from hydrogen ions, hydroxyl ions, pyrophosphate, charge transfer, and heat. In some embodiments, the sensor detects a change in pH. In some embodiments, the sensor comprises a field effect transistor (FET). In some embodiments, the sensor comprises a chemical field effect transistor (chemFET). In some embodiments, the sensor comprises an ion-sensitive field effect transistor (ISFET).

In some embodiments, the nucleic acid amplification reaction is conducted in a single reaction mixture containing a single continuous liquid phase. As used herein, a “single continuous liquid phase” provides no substantial compartmentalization. For example, a single continuous liquid phase comprises only an aqueous phase. In some embodiments, a single continuous liquid phase lacks an oil phase. In some embodiments, a single continuous liquid phase lacks discrete aqueous phase droplets enclosed in an oil phase. In some embodiments, a single continuous liquid phase does not provide compartmentalization for multiple nucleic acid amplification reactions occurring in a single reaction vessel. In some embodiments, multiple nucleic acid amplification reactions occur in an aqueous phase in a single reaction vessel. In some embodiments, a single continuous liquid phase contains multiple nucleic acid amplification reactions that include multiple target polynucleotides (e.g., templates) having the same or different sequences.

In some embodiments, the nucleic acid amplification reaction is conducted in a single reaction mixture containing an emulsion comprising two immiscible liquid phases. In some embodiments, two immiscible liquid phases are mixed together to make the emulsion. In some embodiments, one of the liquid phases is dispersed in the other. In some embodiments, methods for nucleic acid amplification can be conducted in a discontinuous liquid phase. In some embodiments, methods for nucleic acid amplification can be conducted in a water-in-oil emulsion that provides compartmentalization (micro-reactors).

In some embodiments, the disclosure relates generally to compositions, and related methods, systems, kits and apparatuses, comprising a nucleic acid amplification reaction mixture distributed into one or more reaction vessels. In some embodiments, a single reaction vessel contains an amplification reaction mixture. Non-limiting examples of a single reaction vessel include a tube, inner wall of a tube, well, microwell, reaction chamber, groove, channel reservoir, flowcell, or similar structures.

In some embodiments, the nucleic acid amplification reaction mixture can be distributed directly into two or more reaction chambers arranged in an array. In some embodiments, the reaction chambers can be arranged in a grid or array. For example, an array can include two or more reaction chambers. Multiple reaction chambers can be arranged randomly or in an ordered array. An ordered array can include reaction chambers arranged in a single row, or in a two-dimensional grid with rows and columns. In some embodiments, the array can include one or more reaction chambers on a support. A reaction chamber can have walls and a bottom that define width and depth. The dimensions of a reaction chamber can be sufficient to permit deposition of reagents or for conducting nucleic acid amplification reactions. A reaction chamber can have any shape including cylindrical, polygonal or a combination of different shapes. Any wall of a reaction chamber can have a smooth or irregular surface. A reaction chamber can have a bottom with a planar, concave or convex surface. The bottom and side walls of a reaction chamber can comprise the same or different material and/or can be coated with a chemical group that can react with a biomolecule such as nucleic acids, proteins or enzymes.

An array can include any number of reaction chambers for depositing reagents and conducting numerous individual reactions. For example, an array can include at least 256 reaction chambers, or at least 256,000, or at least 1-3 million, or at least 3-5 million, or at least 5-7 million, or at least 7-9 million, at least 9-11 million, at least 11-13 million reaction chambers, or even high density including 13-700 million reaction chambers or more. Reaction chambers arranged in a grid can have a center-to-center distance between adjacent reaction chambers (e.g., pitch) of less than about 10 microns, or less than about 5 microns, or less than about 1 microns, or less than about 0.5 microns.

An array can include reaction chambers having any width and depth dimensions. For example, a reaction chamber can have dimensions to accommodate a single microparticle (e.g., microbead) or multiple microparticles. A reaction chamber can hold about 0.001-100 picoliters of aqueous volume.

In some embodiments, at least one reaction chamber can be coupled to one or more sensors or can be fabricated above one or more sensors. A reaction chamber that is coupled to a sensor can provide confinement of reagents deposited therein so that products from a reaction can be detected by the sensor. A sensor can detect changes in products from any type of reaction, including any nucleic acid reaction such as primer extension, amplification or nucleotide incorporation reactions, within the reaction chamber. A sensor can detect changes in ions (e.g., hydrogen ions), protons, phosphate groups such as pyrophosphate groups. A sensor can detect at least one by product of nucleotide incorporation, including pyrophosphate, hydrogen ions, charge transfer, or heat. In some embodiments, at least one reaction chamber can be coupled to one or more field effect transistor (FET), including for example an ion sensitive field effect transistor (ISFET). Examples of an array of reaction chambers coupled to ISFET sensors can be found at U.S. Pat. No. 7,948,015, and U.S. Ser. No. 12/002,781, hereby incorporated by reference in their entireties. Other examples of sensors that detect byproducts of a nucleotide incorporation reaction can be found, for example, in Pourmand et al, Proc. Natl. Acad. Sci., 103: 6466-6470 (2006); Purushothaman et al., IEEE ISCAS, IV-169-172; Anderson et al, Sensors and Actuators B Chem., 129: 79-86 (2008); Sakata et al., Angew. Chem. 118:2283-2286 (2006); Esfandyapour et al., U.S. Patent Publication No. 2008/01666727; and Sakurai et al., Anal. Chem. 64: 1996-1997 (1992).

In some embodiments, a single reaction vessel can contain any one or any combination of reagents for conducting a nucleic acid amplification reaction, including a plurality of particles (e.g., individual particles in the plurality are attached with a plurality of a capture primer), a plurality of target polynucleotides, a plurality of solution-phase primers, one or more polymerases, divalent cations and/or a plurality of nucleotides. In some embodiments, nucleic acid amplification reaction can further include any combination of a recombinase, recombinase accessory proteins, ATP, co-factors and/or one or more sieving agent or diffusion reducing agent. In some embodiments, any combination of reagents for conducting a nucleic acid amplification reaction can be deposited into a reaction vessel in any order, including sequentially or substantially simultaneously or a combination of both. In some embodiments, a nucleic acid amplification reaction can be conducted in a single reaction vessel comprising a single continuous liquid phase or an emulsion.

In some embodiments, the disclosure relates generally to compositions, as well as related systems, methods, kits and apparatuses, comprising a plurality of target polynucleotides. As used herein, the terms “target polynucleotide” and “template polynucleotide” are used interchangeably. In some embodiments, the target polynucleotides comprise single-stranded or double-stranded polynucleotides, or a mixture of both. In some embodiments, the target polynucleotides include polynucleotides having the same sequence or a mixture of different sequences. In some embodiments, the target polynucleotides include polynucleotides having the same or different lengths. In some embodiments, the plurality of target polynucleotides includes about 2-10, or about 10-50, or about 50-100, or about 100-500, or about 500-1,000, or about 1,000-5,000, or about 10³-10⁶, or about 10⁶-10¹⁰ or more target polynucleotide molecules. In some embodiments, the target polynucleotides comprise polymers of deoxyribonucleotides, ribonucleotides, and/or analogs thereof. In some embodiments, the target polynucleotides comprise naturally-occurring, synthetic, recombinant, cloned, fragmented, non-fragmented, amplified, unamplified or archived (e.g., preserved) forms. In some embodiments, the target polynucleotides comprise DNA, cDNA, RNA, RNA/DNA, or nucleic acid analogs. In some embodiments, the target polynucleotides comprise mRNA, miRNA, rRNA or tRNA.

In some embodiments, the target polynucleotides have one or both ends joined to a nucleic acid adaptor. For example, the first end of a target polynucleotide can be joined to a first nucleic acid adaptor. Optionally, the second end of the target polynucleotide can be joined to a second nucleic acid adaptor. The first and second adaptors can have the same or different sequence. In some embodiments, at least a portion of the first or second nucleic acid adaptor can hybridize to the capture primer, fusion primer, solution-phase primer, amplification primer or sequencing primers. In some embodiments, the first and/or second nucleic acid adaptor comprises an identifier sequence, such as a barcode sequence.

In some embodiments, target polynucleotides can be compatible for use in any type of sequencing platform including chemical degradation, chain-termination, sequence-by-synthesis, pyrophosphate, massively parallel, ion-sensitive, and single molecule sequencing platforms.

In some embodiments, the disclosure relates generally to compositions, as well as related systems, methods, kits and apparatuses, comprising target polynucleotides isolated from a biological sample, including a biological fluid, cell culture or solid tissue. In some embodiments, the biological sample includes a biological fluid obtained from blood, serum, plasma, saliva, sputum, sweat, tears, lavage fluid, amniotic fluid, cerebrospinal fluid, ascites, urine, semen and the like. For example, blood, serum and plasma include fractions or processed portions thereof. Optionally, the target polynucleotide can be extracted from a formalin fixed paraffin-embedded (FFPE) sample. In some embodiments, the biological fluid is added directly to a reaction vessel along with various reagents for conducting a nucleic acid amplification reaction. That is, the cells contained within the biological fluid are lysed in the nucleic acid amplification reaction mixture to release the target polynucleotides. In some embodiments, a separate cell lysis step is not practiced, or a lysis step is conducted prior to the nucleic acid amplification step. In some embodiments, the target polynucleotide is from cell-free DNA or RNA contained within the biological fluid. In some embodiments, a biological sample includes a biological fluid or solid tissue obtained by biopsy, swab, or smear. In some embodiments, the solid tissue includes healthy or diseased tissue, or a mixture of both.

In some embodiments, the disclosure relates generally to compositions, as well as related systems, methods, kits and apparatuses, comprising target polynucleotides and at least one adaptor.

In some embodiments, the target polynucleotides are joined or appended to at least one adaptor. In some embodiments, the target polynucleotides lack any adaptor. In some embodiments, one or more adaptors can be joined to the target polynucleotide by ligation. In some embodiments, a tailed amplification primer can be used in a PCR reaction to append one or more adaptors to a target polynucleotide, where the tailed amplification primer includes the sequence of one or more adaptors.

In some embodiments, the adaptor comprises a nucleic acid, including DNA, RNA, RNA/DNA molecules, or analogs thereof. In some embodiments, the adaptor can include one or more deoxyribonucleoside or ribonucleoside residues. In some embodiments, the adaptor can be single-stranded or double-stranded nucleic acids, or can include single-stranded and/or double-stranded portions. In some embodiments, the adaptor can have any structure, including linear, hairpin, forked (Y-shaped), or stem-loop.

In some embodiments, the adaptor can have any length, including fewer than 10 bases in length, or about 10-20 bases in length, or about 20-50 bases in length, or about 50-100 bases in length, or longer.

In some embodiments, the adaptor can have any combination of blunt end(s) and/or sticky end(s). In some embodiments, at least one end of the adaptor can be compatible with at least one end of a nucleic acid fragment. In some embodiments, a compatible end of the adaptor can be joined to a compatible end of a nucleic acid fragment. In some embodiments, the adaptor can have a 5′ or 3′ overhang end.

In some embodiments, the adaptor can have a 5′ or 3′ overhang tail. In some embodiments, the tail can be any length, including 1-50 or more nucleotides in length.

In some embodiments, the adaptor can include an internal nick. In some embodiments, the adaptor can have at least one strand that lacks a terminal 5′ phosphate residue. In some embodiments, the adaptor lacking a terminal 5′ phosphate residue can be joined to a nucleic acid fragment to introduce a nick at the junction between the adaptor and the nucleic acid fragment.

In some embodiments, the adaptor can include a nucleotide sequence that is identical or complementary to any portion of the target polynucleotide, capture primer, fusion primer, solution-phase primer, amplification primer, or a sequencing primer.

In some embodiments, the adaptor can include a unique identifier sequence (e.g., barcode sequence). In some embodiments, a barcoded adaptor can be used for constructing a multiplex library of target polynucleotides. In some embodiments, the barcoded adaptors can be appended to a target polynucleotide and used for sorting or tracking the source of the target polynucleotide. In some embodiments, one or more barcode sequences can allow identification of a particular adaptor among a mixture of different adaptors having different barcodes sequences. For example, a mixture can include 2, 3, 4, 5, 6, 7-10, 10-50, 50-100, 100-200, 200-500, 500-1000, or more different adaptors having unique barcode sequences.

In some embodiments, the adaptor can include degenerate sequences. In some embodiments, the adaptor can include one or more inosine residues.

In some embodiments, the adaptor can include at least one scissile linkage. In some embodiments, the scissile linkage can be susceptible to cleavage or degradation by an enzyme or chemical compound. Optionally, the adaptor includes at least one uracil base. In some embodiments, the adaptor can include at least one phosphorothiolate, phosphorothioate, and/or phosphoramidate linkage.

In some embodiments, the adaptor can include any type of restriction enzyme recognition sequence, including type I, type II, type Hs, type IIB, type III, type IV restriction enzyme recognition sequences, or recognition sequences having palindromic or non-palindromic recognition sequences.

In some embodiments, the adaptor can include a cell regulation sequences, including a promoter (inducible or constitutive), enhancers, transcription or translation initiation sequence, transcription or translation termination sequence, secretion signals, Kozak sequence, cellular protein binding sequence, and the like.

In some embodiments, a target polynucleotide comprises a universal adaptor. A universal adaptor is a sequence suitable for use as a primer hybridization site that is joined or appended to multiple target polynucleotides in a sample, such as the majority of or substantially all of the multiple target nucleotides in a sample, the multiple target polynucleotides not having identical sequences.

In some embodiments, the disclosure relates generally to methods, as well as related systems, compositions, kits and apparatuses, comprising particles, microparticles or beads. One skilled in the art will appreciate that the methods, as well as related systems, compositions, kits and apparatuses, can include a first, and optionally a second, third, fourth, fifth, sixth, seventh, eighth, ninth, tenth, eleventh, or more different sets of particles. In some embodiments, different capture primers are attached (e.g., immobilized) to the different sets of particles. For example, a first set of particles is attached with a plurality of a first capture primer, and a second set of particles is attached with a plurality of a second capture primer, and so on with the other sets of particles. In some embodiments, the particles can be attached with a plurality of one capture primers having the same sequence, or can be attached a plurality of two or more different capture primers having different sequences. The capture primer can selectively bind (hybridize) to at least a portion of the target polynucleotide or an adaptor sequence.

In some embodiments, the plurality of particles can be solid, or can have an outer surface and an interior surface. The plurality of particles can be porous, semi porous or non-porous. The plurality of particles can have cavitation or pores, or can include three-dimensional scaffolds. In some embodiments, the plurality of particles can be Ion Sphere™ particles (from Ion Torrent, part of Life Technologies, Carlsbad, Calif.).

In some embodiments, the plurality of particles comprise a polymer material. For example, the plurality of particles comprise a gel, hydrogel or acrylamide polymers. In some embodiments, the plurality of particles can have any shape that is spherical, hemispherical, cylindrical, barrel-shaped, toroidal, rod-like, disc-like, conical, triangular, cubical, polygonal, tubular, wire-like or irregular.

In some embodiments, the particles can be any size that can fit into a reaction chamber. For example, the particles can be small enough to fit one particle in a reaction chamber. In some embodiments, the particles can be small enough so that more than one particle can fit in a reaction chamber. In some embodiments, the smallest cross-sectional length of a particle (e.g., diameter) can be about 50 microns or less, or about 10 microns or less, or about 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers).

In some embodiments, the particles can be attached with a plurality of at least 1,000 oligonucleotide primers, or about 1,000-10,000 oligonucleotide primers, or about, 10,000-50,000 oligonucleotide primers, or about 50,000-75,000 oligonucleotide primers, or about 75,000-100,000 oligonucleotide primers, or more.

In some embodiments, the exterior surface and/or the interior scaffold of a particle can be attached with one or more capture primers. Optionally, the capture primer includes a universal priming sequence or site. Optionally, the capture primers can include any or any combination of an amplification primer sequence, a sequencing primers sequence, unique identifier sequence and/or a source identifier sequence.

In some embodiments, a particle surface, and optionally interior scaffold, can be coated with an acrylamide, carboxylic or amine compound for attaching a nucleic acid (e.g., capture primer). In some embodiments, an amino-modified capture primer can be attached to a particle surface that is coated with a carboxylic acid. In some embodiments, an amino-modified capture primer can be reacted with ethyl (dimethylaminopropyl) carbodiimide (EDC) or EDAC for attachment to a carboxylic acid coated surface (with or without N-hydoxysuccinimide (NETS)). A capture primer can be immobilized to an acrylamide compound coating on a particle surface. Particles can be coated with an avidin-like compound (e.g., streptavidin) for binding biotinylated capture primers.

In some embodiments, the disclosure relates generally to methods, as well as related systems, compositions, kits and apparatuses, comprising one or more capture primers attached to a support, including a particle or planar-like surface. In some embodiments, the methods, as well as related systems, compositions, kits and apparatuses, comprise at least a first set of particles attached to a plurality of a first capture primer. In some embodiments, the methods, as well as related systems, compositions, kits and apparatuses, additionally include a second particle attached to a plurality of a second capture primer. In some embodiments, the compositions, as well as related systems, methods, kits and apparatuses, comprise additional capture primers, including a third, fourth, fifth, sixth, seventh, eighth, ninth, tenth, eleventh, or more different capture primers. In some embodiments, the capture primers of one set have a different nucleotide sequence compared to the capture primers of a different set. In some embodiments, the capture primers comprise polymers of deoxyribonucleotides, ribonucleotides, and/or analogs thereof. In some embodiments, the capture primers comprise naturally-occurring, synthetic, recombinant, cloned, amplified, or unamplified forms. In some embodiments, the capture primers comprise DNA, cDNA, RNA, chimeric RNA/DNA, or nucleic acid analogs. In some embodiments, the capture primers comprise single-stranded oligonucleotides. In some embodiments, the capture primers comprise a random or degenerate sequence. In some embodiments, at least one portion of the capture primer comprises a sequence that can selectively bind (hybridize) to at least one portion of the target polynucleotide. For example, the capture primer includes a gene-specific or target-specific sequence, and hybridizes to one strand of the target polynucleotide. In some embodiments, at least one portion of the capture primer hybridizes to an adaptor joined to the target polynucleotide, or the junction between the adaptor and target polynucleotide. In some embodiments, at least one portion of the capture primers comprises a sequence that can hybridize with at least one portion of a fusion primer. In some embodiments, at least one portion of the capture primers comprises a sequence that is identical or is partially or fully complementary to a portion of a target polynucleotide, an adaptor, or a fusion primer. For example, the 3′ region of the capture primers comprises a sequence that is identical or is complementary to a portion of a target polynucleotide, an adaptor, or a fusion primer. A “polynucleotide-specific capture primer” refers to a capture primer that is capable of hybridizing to a portion of a target polynucleotide under conditions suitable for primer extension, amplification, and/or sequencing.

Optionally, the capture primer includes at least one universal sequence (amplification or sequencing primer sequence) and/or at least one unique identifier sequence (e.g., a barcode sequence).

In some embodiments, the 5′ or 3′ end of the capture primer can be modified for attachment to the support. For example, a 5′ or 3′ end can be modified to include an amino group that can bind to a carboxylic acid compound on the support. In another example, a 5′ end can include a phosphate group for reacting with an amine-coated surface in the presence of a carbodiimide (e.g., water soluble carbodiimide).

In some embodiments, the capture primers comprise forward amplification primers.

In some embodiments, the 3′ end of the capture primer is extendible in a primer extension reaction. Optionally, the 3′ end of the capture primer includes a 3′ OH group. In some embodiments, the capture primer has a blocking moiety that prevents extension in a primer extension reaction.

In some embodiments, the capture primers can be any length, including about 2-100 nucleotides, or about 5-10 nucleotides, or about 10-25 nucleotides, or about 25-40 nucleotides, or about 40-55 nucleotides, or about 55-70 nucleotides, or about 70-85 nucleotides, or about 85-100 nucleotides, or longer.

In some embodiments, the capture primers include at least one linkage or base that is resistant to degradation by an exonuclease or endonuclease. For example, the fusion primers and reverse amplification primers can include at least one phosphorothioate linkage or a 3′-3′ end linkage for exonuclease resistance, or at least one 2′ fluoro or 2′O-methyl modification for endonuclease resistance. In some embodiments, the capture primer include at least one phosphorothiolate, phosphorothioate, and/or phosphoramidate linkage.

In some embodiments, the disclosure relates generally to methods, and related compositions, systems, kits and apparatuses, that also include a solution-phase primer (e.g., soluble primer). In some embodiments, at least one portion of a solution-phase primer comprises a sequence that can selectively bind (hybridize) with at least one portion of the target polynucleotide, or an adaptor joined to the target polynucleotide, a capture primer, or a fusion primer. In some embodiments, at least one portion of the solution-phase primer comprises a sequence that is substantially identical or is complementary to a portion of a target polynucleotide, an adaptor, a capture primer, or a fusion primer. In some embodiments, at least a portion of the solution-phase primer can be partially or fully complementary to a portion of the target polynucleotide or to the nucleic acid adaptor or to a junction between the target polynucleotide and adaptor.

In some embodiments, the solution-phase primer comprises single-stranded oligonucleotides. In some embodiments, the solution-phase primer is not attached to any support (e.g., a particle or planar-like surface). Optionally, the solution-phase primer comprises a reverse amplification primer.

Optionally, the solution-phase primer includes a universal priming sequence or site.

Optionally, the solution-phase primers can include any one or any combination of an amplification primer sequence, a sequencing primers sequence, unique identifier sequence and/or a source identifier sequence.

Optionally, the solution-phase primer comprises two regions: a first region that is identical or complementary to an adaptor attached to the template polynucleotide and a second region that is identical or complementary to a blocking primer. In some embodiments, the first region is also identical or complementary to an enrichment primer and/or a sequencing primer.

Optionally, the solution-phase primer includes a binding partner. Optionally, the binding partner comprises biotin.

Optionally, the 5′ end of the solution-phase primer can include a sequence that is not contained in, or is not complementary to, a sequence in the target polynucleotide. For example, the solution-phase primer can be a tailed primer. In some embodiments, the sequence that is not contained in, or is not complementary to, a sequence in the target polynucleotide is a sequence comprising at least one uridine. In some embodiments, the sequence that is not contained in, or is not complementary to, a sequence in the target polynucleotide is a sequence comprising at least two uridines. In some embodiments, the sequence that is not contained in, or is not complementary to, a sequence in the target polynucleotide is a sequence comprising at least one uridine.

Optionally, the 5′ end of the solution-phase primer can include a sequence that is not complementary to a sequence in the target polynucleotide and that is complementary to the 3′ end of the solution-phase primer. In some embodiments, the sequence that is not complementary to a sequence in the target polynucleotide and that is complementary to the 3′ end of the solution-phase primer comprises at least one uridine. Optionally, the 5′ end of the solution-phase primer can include a sequence that is not complementary to a sequence in the target polynucleotide and that is complementary to another region of the solution-phase primer. The region can be a region including, e.g., the first, second, third, fourth, fifth, sixth, seventh, eighth, ninth, tenth, eleventh, twelfth, thirteenth, fourteenth, fifteenth, sixteenth, seventeenth, eighteenth, nineteenth, and/or twentieth nucleotide from the 3′ end of the solution-phase primer. In some embodiments, the sequence that is not complementary to a sequence in the target polynucleotide and that is complementary to another region of the solution-phase primer is a sequence comprising at least one uridine.

In some embodiments, the solution-phase primer comprises polymers of deoxyribonucleotides, ribonucleotides, and/or analogs thereof. In some embodiments, the solution-phase primer comprises naturally-occurring, synthetic, recombinant, cloned, amplified, or unamplified forms. In some embodiments, the solution-phase primer comprises DNA, cDNA, RNA, chimeric RNA/DNA, or nucleic acid analogs. In some embodiments, the solution-phase primer comprises a random or degenerate sequence.

In some embodiments, the 3′ end of the solution-phase primer is extendible in a primer extension reaction. Optionally, the 3′ end of the solution-phase primer includes a 3′ OH group. In some embodiments, the solution-phase primer has a blocking moiety that prevents extension in a primer extension reaction.

In some embodiments, the solution-phase primer can be any length, including about 2-100 nucleotides, or about 5-10 nucleotides, or about 10-25 nucleotides, or about 25-40 nucleotides, or about 40-55 nucleotides, or about 55-70 nucleotides, or about 70-85 nucleotides, or about 85-100 nucleotides, or longer.

In some embodiments, the solution-phase primers include at least one linkage or base that is resistant to degradation by an exonuclease or endonuclease. For example, the fusion primers and reverse amplification primers can include at least one phosphorothioate linkage or a 3′-3′ end linkage for exonuclease resistance, or at least one 2′ fluoro or 2′O-methyl modification for endonuclease resistance.

In some embodiments, the solution-phase primer comprises a binding partner or affinity moiety (e.g., biotin) for affinity-based enrichment of the amplified polynucleotides. Optionally, the solution-phase primer includes at least one universal sequence (amplification or sequencing primer sequence) and/or at least one unique identifier sequence (e.g., a barcode sequence). In some embodiments, a solution-phase primer comprising a binding partner or affinity moiety is referred to as an “enrichment primer.”

In some embodiments, the 5′ or 3′ region of the solution-phase primer includes a sequence that is substantially non-complementary to a portion of the target polynucleotide or to a portion of any adaptor (e.g., a tailed primer).

A blocking primer is a solution-phase primer that specifically hybridizes to a sequence, such as an adaptor sequence, and thus reduces or eliminates mis-priming by a non-complementary solution-phase primer to the blocked sequence. For example, a blocking primer can bind an adaptor sequence on a primer-dimer or on a bead-primer-dimer, to reduce mis-priming. A blocking primer may be used in conjunction with an enrichment primer to reduce or prevent hybridization of the enrichment primer to a non-complementary sequence, thus improving enrichment of the desired product. In some embodiments, a blocking primer may be used in conjunction with a sequencing primer to reduce unwanted sequencing products resulting from mis-priming.

In some embodiments, the disclosure relates generally to methods, and related compositions, systems, kits and apparatuses, comprise a fusion primer. In some embodiments, a solution-phase primer is a fusion primer. In some embodiments, the fusion primer is provided as an additional primer, which may be an amplification primer (such as a solution-phase amplification primer), an enrichment primer (such as a solution-phase enrichment primer), or a blocking primer (such as a solution-phase blocking primer). For example, a fusion primer can have two regions. The first region can hybridize to at least a portion of one strand of the target polynucleotide or an adaptor attached to the target polynucleotide. The first region may also be identical or complementary to a sequencing primer and/or an enrichment primer. The second region, in some embodiments, is identical or complementary to a blocking primer. In some embodiments, the fusion primer comprises a universal sequence. The fusion primer may also comprise an identifier sequence (e.g., a barcode sequence), which may be unique. In some embodiments, the fusion primer comprises an extendible 3′ end that provides an initiation site for nucleotide polymerization. In some embodiments, at least a portion of the fusion primer can be partially or fully complementary to a portion of the target polynucleotide or an adaptor attached thereto.

In some embodiments, any oligonucleotide primer (e.g., capture, reverse solution-phase or fusion primer) can be compatible for use in any type of sequencing platform including chemical degradation, chain-termination, sequence-by-synthesis, pyrophosphate, massively parallel, ion-sensitive, and single molecule platforms. In some embodiments, any oligonucleotide primer can be compatible for use in any type of sequencing including: sequencing by oligonucleotide probe ligation and detection (e.g., SOLiD™), probe-anchor ligation sequencing (e.g., Complete Genomics or Polonator™), sequence-by-synthesis (e.g., Illumina), pyrophosphate sequencing (e.g., 454 Life Sciences), ion-sensitive sequencing (e.g., Personal Genome Machine (PGM™) and Ion Proton™ Sequencer, both from Ion Torrent Systems, Inc.) and single molecule sequencing platforms (e.g., Helicos™).

In some embodiments, the disclosure relates generally to compositions, as well as related systems, methods, kits and apparatuses, comprising one or more polymerases. In some embodiments, the compositions (and related methods, systems, kits and apparatuses) includes one type, or a mixture of different types of polymerases. In some embodiments, the polymerase includes any enzyme, or fragment or subunit of thereof, that can catalyze polymerization of nucleotides and/or nucleotide analogs. In some embodiments, the polymerase requires a nucleic acid having an extendible 3′ end. For example, the polymerase can require a terminal 3′ OH of a nucleic acid primer to initiate nucleotide polymerization.

The polymerase comprises any enzyme that can catalyze the polymerization of nucleotides (including analogs thereof) into a nucleic acid strand. Typically but not necessarily such nucleotide polymerization can occur in a template-dependent fashion. In some embodiments, the polymerase can be a high fidelity polymerase. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide, such as, for example, a reporter enzyme or a processivity-enhancing domain. Typically, the polymerase comprises one or more active sites at which nucleotide binding and/or catalysis of nucleotide polymerization can occur. In some embodiments, the polymerase includes or lacks other enzymatic activities, such as for example, 3′ to 5′ exonuclease activity or 5′ to 3′ exonuclease activity. In some embodiments, the polymerase can be isolated from a cell, or generated using recombinant DNA technology or chemical synthesis methods. In some embodiments, the polymerase can be expressed in prokaryote, eukaryote, viral, or phage organisms. In some embodiments, the polymerase can be post-translationally modified proteins or fragments thereof.

In some embodiments, the polymerase can be a DNA polymerase and include without limitation bacterial DNA polymerases, eukaryotic DNA polymerases, archaeal DNA polymerases, viral DNA polymerases and phage DNA polymerases.

In some embodiments, the polymerase can be a replicase, DNA-dependent polymerase, primases, RNA-dependent polymerase (including RNA-dependent DNA polymerases such as, for example, reverse transcriptases), a thermo-labile polymerase, or a thermo-stable polymerase. In some embodiments, the polymerase can be any Family A or B type polymerase. Many types of Family A (e.g., E. coli Pol I), B (e.g., E. coli Pol II), C (e.g., E. coli Pol III), D (e.g., Euryarchaeotic Pol II), X (e.g., human Pol beta), and Y (e.g., E. coli UmuC/DinB and eukaryotic RAD30/xeroderma pigmentosum variants) polymerases are described in Rothwell and Watsman 2005 Advances in Protein Chemistry 71:401-440. In some embodiments, a polymerase can be a T3, T5, T7, or SP6 RNA polymerase.

In some embodiments, the polymerase comprises a heat-stable polymerase. In some embodiments, the polymerase comprises a heat-labile polymerase. In some embodiments, the polymerase comprises a low fidelity polymerase. In some embodiments, the polymerase comprises a high fidelity polymerase.

In some embodiments, the polymerase can lack 5′-3′ exonuclease activity. In some embodiments, the polymerase can have strand-displacement activity.

In some embodiments, the archaeal DNA polymerase, can be, without limitation, a thermostable or thermophilic DNA polymerase such as, for example: a Bacillus subtilis (Bsu) DNA polymerase I large fragment; a Thermus aquaticus (Taq) DNA polymerase; a Thermus filiformis (Tfi) DNA polymerase; a Phi29 DNA polymerase; a Bacillus stearothermophilus (Bst) DNA polymerase; a Thermococcus sp. 9° N-7 DNA polymerase; a Bacillus smithii (Bsm) DNA polymerase large fragment; a Thermococcus litoralis (Tli) DNA polymerase or Vent™ (exo-) DNA polymerase (from New England Biolabs); or “Deep Vent” (exo-) DNA polymerase (New England Biolabs). In some embodiments, the polymerase comprises E. coli large fragment DNA polymerase I (e.g., Klenow).

In some embodiments, the disclosure relates generally to compositions, as well as related systems, methods, kits and apparatuses, comprising at least one accessory protein. In some embodiments, the accessory protein can bind single-stranded or double-stranded nucleic acids. Optionally, the accessory protein can mediate loading other proteins (e.g., recombinase) onto a nucleic acid. Optionally, the accessory protein can unwind nucleic acid substrates, relax nucleic acids, resolve nucleic acid structures, hydrolyze nucleic acids (e.g., nuclease), disassemble complexes of nucleic acids and proteins, or disassemble nucleic acid structures. Optionally, the accessory protein can partially or fully denature a double-stranded first or second target nucleic acid. Optionally, the accessory protein can catalyze strand invasion or unwinding. Optionally, the accessory protein comprises a sliding clamp protein. Optionally, the accessory protein can mediate or catalyze its respective activity in a sequence-specific or sequence-independent manner.

In some embodiments, the accessory protein comprises wild-type, mutant, recombinant, fusion, or fragments thereof.

In some embodiments, an accessory protein comprises a multimeric protein complex. Optionally, the multimeric protein complex comprises 2, 3, 4, 5, 6, 7, 8, or more subunits. Optionally, the multimeric accessory protein complex comprises a homo-meric or hetero-meric protein complex.

In some embodiments, the accessory proteins can originate from any bacteriophage including a myoviral phage. The accessory proteins can originate from bacteriophage T2, T4, T5 or T7. The accessory proteins can originate from any prokaryote, archaeon, bacterium (e.g., E. coli), eukaryote, or mammal (e.g., human).

In some embodiments, the accessory proteins comprise a single-stranded binding protein including myoviral gp32 (e.g., T4 or RB69), Sso SSB from Sulfolobus solfataricus, MjA SSB from Methanococcus jannaschii, or E. coli SSB protein.

In some embodiments, the single reaction mixture comprises a mixture of different accessory proteins that originate from the same or different species. Optionally, the single reaction mixture comprises a mixture of different accessory proteins that originate from the same or different species as a recombinase enzyme.

In some embodiment, the accessory protein comprises a single-stranded binding protein (e.g., SSB or gp32), recombinase (e.g., recA or uvsX), recombinase loading protein (e.g., uvsY), helicase (e.g., uvsW), or topoisomerase.

In some embodiments, the disclosure relates generally to compositions, as well as related systems, methods, kits and apparatuses, comprising at least one enzyme that catalyzes homologous recombination. For example an enzyme that catalyzes homologous recombination can bind an oligonucleotide primer (e.g., capture primer, reverse solution-phase primer or fusion primer) to form a nucleoprotein complex. In some embodiments, a homologous recombination enzyme can catalyze strand invasion by forming a nucleoprotein complex and binding to a homologous portion of a double-stranded polynucleotide to form a recombination intermediate having a triple-strand structure (e.g., D-loop formation).

In some embodiments, the enzyme that catalyzes homologous recombination comprises wild-type, mutant, recombinant, fusion, or fragments thereof. In some embodiments, a homologous recombination enzyme comprises at least a portion of a recombinase enzyme from any organism, including bacteriophages (e.g., uvsX, such as bacteriophage T4 uvsX), bacteria (e.g., recA, such as Escherichia coli recA), or eukaryotes (e.g., RAD51, such as human or Saccharomyces cerevisiae RAD51).

In some embodiments, the enzyme that catalyzes homologous recombination comprises a recombinase enzyme, such as a member of the uvsX/recA/RAD51 family.

In some embodiments, the recombinase can form a nucleoprotein complex by binding a capture primer. Optionally, the nucleoprotein complex further includes a target polynucleotide, where a portion of the capture primer hybridizes to a portion of the target polynucleotide. Optionally, the target polynucleotide comprises a double-stranded polynucleotide molecule. Optionally, the recombinase can partially or fully denature the double-stranded first target nucleic acid.

In some embodiments, the recombinase can form a nucleoprotein complex by binding a solution-phase primer. Optionally, the nucleoprotein complex further includes a target polynucleotide, where a portion of the solution-phase primer hybridizes to a portion of the target polynucleotide. Optionally, the target polynucleotide comprises a double-stranded polynucleotide molecule. Optionally, the recombinase can partially or fully denature the double-stranded second target nucleic acid.

In some embodiments, the recombination enzyme comprises at least a portion of a recombinase enzyme from any organism, including Escherichia coli (e.g., recA), human (e.g., RAD51), or bacteriophage T4 (e.g., uvsX) (U.S. Pat. No. 5,223,414 to Zarling, U.S. Pat. Nos. 5,273,881 and 5,670,316 both to Sena, and U.S. Pat. Nos. 7,270,981, 7,399,590, 7,435,561, 7,666,598, 7,763,427, 8,017,339, 8,030,000, 8,062,850, and 8,071,308).

In some embodiments, the disclosure relates generally to compositions, as well as related systems, methods, kits and apparatuses, comprising at least one accessory protein that improves the activity of a recombinase enzyme (see, e.g., U.S. Pat. No. 8,071,308 granted to Piepenburg, et al.). In some embodiments, an accessory protein can bind single strands of nucleic acids, or can load a recombinase onto a nucleic acid. In some embodiments, an accessory protein comprises wild-type, mutant, recombinant, fusion, or fragments thereof. In some embodiments, an accessory protein and a recombinase enzyme can originate from any combination of the same or different species. Accessory proteins can originate from any bacteriophage including a myoviridae phage. Examples of a myoviridae phage include T4, T2, T6, Rb69, Aeh1, KVP40, Acinetobacter phage 133, Aeromonas phage 65, cyanophage P-SSM2, cyanophage PSSM4, cyanophage S-PM2, Rb14, Rb32, Aeromonas phage 25, Vibrio phage nt-1, phi-1, Rb16, Rb43, Phage 31, phage 44RR2.8t, Rb49, phage Rb3, and phage LZ2 (U.S. Pat. No. 8,071,308 granted to Piepenburg). Accessory proteins can originate from any bacterial species, including Escherichia coli, Sulfolobus (e.g., S. solfataricus) or Methanococcus (e.g., M. jannaschii).

In some embodiments, the disclosure relates generally to compositions, as well as related systems, methods, kits and apparatuses, comprising at least one co-factor for polymerase or recombinase activity. In some embodiments, a co-factor comprises one or more divalent cation. Examples of divalent cations include magnesium, manganese and calcium.

In some embodiments, the disclosure relates generally to compositions, as well as related systems, methods, kits and apparatuses, comprising at least one co-factor for polymerase or recombinase assembly on nucleic acids or for homologous nucleic acid pairing. In some embodiments, a co-factor comprise any form of ATP including ATP and ATPγS.

In some embodiments, the disclosure relates generally to compositions, as well as related systems, methods, kits and apparatuses, comprising at least one co-factor that regenerates ATP. For example, a co-factor comprises an enzyme system that converts ADP to ATP. In some embodiments, a co-factor comprises phosphocreatine and/or creatine kinase.

In some embodiments, the disclosure relates generally to compositions, as well as related systems, methods, kits and apparatuses, comprising one or more nucleotides. In some embodiments, the compositions (and related methods, systems, kits and apparatuses) includes one type, or a mixture of different types of nucleotides. A nucleotide comprises any compound that can bind selectively to, or can be polymerized by, a polymerase. Typically, but not necessarily, selective binding of the nucleotide to the polymerase is followed by polymerization of the nucleotide into a nucleic acid strand by the polymerase. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. In some embodiments, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In some embodiments, one or more phosphorus atoms in the chain can be part of a phosphate group having P and O. In some embodiments, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In some embodiments, the phosphorus atoms in the chain can have side groups having O, BH₃, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

Some examples of nucleotides that can be used in the disclosed compositions (and related methods, systems, kits and apparatuses) include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. In some embodiments, a nucleotide can include a purine or pyrimidine base, including adenine, guanine, cytosine, thymine, uracil or inosine. In some embodiments, a nucleotide includes dATP, dGTP, dCTP, dTTP and dUTP.

In some embodiments, the nucleotide is unlabeled. In some embodiments, the nucleotide comprises a label and referred to herein as a “labeled nucleotide”. In some embodiments, the label can be in the form of a fluorescent dye attached to any portion of a nucleotide including a base, sugar or any intervening phosphate group or a terminal phosphate group, i.e., the phosphate group most distal from the sugar.

In some embodiments, the disclosure relates generally to compositions, as well as related systems, methods, kits and apparatuses, comprising any one or any combination of capture primers, reverse solution-phase primers, fusion primers, target polynucleotides and/or nucleotides that are non-labeled or attached to at least one label. In some embodiments, the label comprises a detectable moiety. In some embodiments, the label can generate, or cause to generate, a detectable signal. In some embodiments, the detectable signal can be generated from a chemical or physical change (e.g., heat, light, electrical, pH, salt concentration, enzymatic activity, or proximity events). For example, a proximity event can include two reporter moieties approaching each other, or associating with each other, or binding each other. In some embodiments, the detectable signal can be detected optically, electrically, chemically, enzymatically, thermally, or via mass spectroscopy or Raman spectroscopy. In some embodiments, the label can include compounds that are luminescent, photoluminescent, electroluminescent, bioluminescent, chemiluminescent, fluorescent, phosphorescent or electrochemical. In some embodiments, the label can include compounds that are fluorophores, chromophores, radioisotopes, haptens, affinity tags, atoms or enzymes. In some embodiments, the label comprises a moiety not typically present in naturally occurring nucleotides. For example, the label can include fluorescent, luminescent or radioactive moieties.

In some embodiments, the disclosure relates generally to compositions, as well as related systems, methods, kits and apparatuses, comprising at least one member of a binding partner. In some embodiments, a binding partners includes two molecules, or portions thereof, which have a specific binding affinity for one another and typically will bind to each other in preference to binding to other molecules. In some embodiments, binding partners include an “affinity moiety” and a “receptor moiety”. Typically but not necessarily some or all of the structure of one member of a specific binding pair is complementary to some or all of the structure possessed by the other member, with the two members being able to bind together specifically by way of a bond between the complementary structures, optionally by virtue of multiple non-covalent attractions.

In some embodiments, molecules that function as binding partners include: biotin (and its derivatives) and its binding partners avidin, streptavidin and their derivatives; His-tags which bind nickel, cobalt or copper; cysteine, histidine, or histidine patch which bind Ni-NTA; maltose which binds with maltose binding protein (MBP); lectin-carbohydrate binding partners; calcium-calcium binding protein (CBP); acetylcholine and receptor-acetylcholine; protein A and binding partner anti-FLAG antibody; GST and binding partner glutathione; uracil DNA glycosylase (UDG) and ugi (uracil-DNA glycosylase inhibitor) protein; antigen or epitope tags which bind to antibody or antibody fragments, particularly antigens such as digoxigenin, fluorescein, dinitrophenol or bromodeoxyuridine and their respective antibodies; mouse immunoglobulin and goat anti-mouse immunoglobulin; IgG bound and protein A; receptor-receptor agonist or receptor antagonist; enzyme-enzyme cofactors; enzyme-enzyme inhibitors; and thyroxine-cortisol. Another binding partner for biotin can be a biotin-binding protein from chicken (Hytonen, et al., BMC Structural Biology 7:8).

In some embodiments, an avidin moiety can include an avidin protein, as well as any derivatives, analogs and other non-native forms of avidin that can bind to biotin moieties. Other forms of avidin moieties include native and recombinant avidin and streptavidin as well as derivatized molecules, e.g. nonglycosylated avidins, N-acyl avidins and truncated streptavidins. For example, avidin moiety includes deglycosylated forms of avidin, bacterial streptavidins produced by Streptomyces (e.g., Streptomyces avidinii), truncated streptavidins, recombinant avidin and streptavidin as well as to derivatives of native, deglycosylated and recombinant avidin and of native, recombinant and truncated streptavidin, for example, N-acyl avidins, e.g., N-acetyl, N-phthalyl and N-succinyl avidin, and the commercial products ExtrAvidin™, Captavidin™, Neutravidin™ and Neutralite Avidin™.

When conducting a nucleic acid amplification reaction with a plurality of target polynucleotides and particles in a single reaction vessel, the target polynucleotides will tend to move randomly from one particle to another due to Brownian motion. Polynucleotide migration increases the incidence of polyclonal amplification on a particle. One technique to reduce polyclonality employs physical barriers to separate one polynucleotide and one particle from other polynucleotides and particles. For example, a water-in-oil emulsion can form aqueous microreactors that provide physical barriers (e.g., compartments) for performing many separate amplification reactions in a single reaction vessel. In some embodiments, Brownian motion in a nucleic acid amplification reaction can be reduced by adding a sieving agent or a diffusion-reducing agent to a single continuous liquid phase. In some embodiments, improving monoclonality of the amplified polynucleotides attached to a particle comprises adding a sieving agent and/or a diffusion-reducing agent to a single continuous liquid phase. In some embodiments, methods for nucleic acid amplification can be conducted with a sieving agent or a diffusion-reducing agent. In some embodiments, a sieving agent or a diffusion-reducing agent can reduce migration of the polynucleotide away from a support (e.g., a particle or bead) during the amplification reaction.

In some embodiments, the disclosure relates generally to compositions, as well as related systems, methods, kits and apparatuses, comprising at least one sieving agent. For example, a nucleic acid amplification reaction mixture can include at least one sieving agent. In some embodiments, a sieving agent comprises any compound that can provide a physical barrier. In some embodiments, a sieving agent provides a molecular sieve. In some embodiments, a sieving agent comprises any compound that can provide a matrix having a plurality of pores that are small enough to reduce the movement/migration of any component of a nucleic acid amplification reaction. For example, components of a nucleic acid amplification reaction include any one or any combination of particles attached with a plurality of capture primers, target polynucleotides, recombinase, polymerase, solution-phase primers, fusion primers, nucleotides, divalent cations, ATP and/or co-factors. For example, a sieving agent can reduce the movement of a target polynucleotide (or a polynucleotide associated with a surface or particle) through the pores. In some embodiments, a sieving agent comprises any compound that can provide a matrix having a plurality of pores that are small enough to slow the movement of a target polynucleotide away from a surface (e.g., particle or planar surface). Thus, a sieving agent can reduce Brownian motion of a target polynucleotide or any other component of a nucleic acid amplification reaction. In some embodiments, a sieving agent can be selected to form pore sizes small enough to reduce movement of a target polynucleotide through the pores, but large enough to permit movement of smaller components in a nucleic acid amplification reaction, such as cations, nucleotides, divalent cations, ATP and co-factors. In some embodiments, the pore size or range of pore sizes can be modulated by increasing or decreasing the concentration of a sieving agent. For example, the molecular weight, intrinsic viscosity and concentration of a sieving agent (or a combination of sieving agents) can be selected to prepare a nucleic acid amplification reaction mixture in a particular solvent (e.g., water) to produce a matrix having a desired pore size or viscosity. In some embodiments, a sieving agent can reduce bulk flow by increasing the viscosity of a nucleic acid amplification reaction mixture. In some embodiments, a sieving agent can be water soluble. In some embodiments, a matrix having a plurality of pores can be prepared by mixing a sieving agent with a solvent (e.g., an aqueous solvent, such as water). In some embodiments, a sieving agent does not interfere with nucleic acid amplification or formation of a recombinase nucleoprotein complex. In some embodiments, conducting a nucleic acid amplification reaction with one or more sieving agents can reduce the movement of a polynucleotide away from a particular particle and can increase the likelihood that the polynucleotide will hybridize with a capture primer attached to a particular particle, where the primer provides an initiation site for nucleotide polymerization which can increase monoclonality of the polynucleotides attached to the particular particle. In some embodiments, the percentage of particle that are attached with a monoclonal population of a polynucleotide can be increased by conducting a nucleic acid amplification reaction with at least one sieving agent.

In some embodiments, a sieving agent comprises a polymer compound. In some embodiments, a sieving agent comprises cross-linked or non-cross linked forms. In some embodiments, a sieving agent comprises linear or branched forms. In some embodiments, a sieving agent comprises charged or neutral forms. In some embodiments, a polymer sieving agent comprises an average molecular weight of about 10,000-2,000,000, or about 12,000-95,000, or about 13,000-100,000. In some embodiments, a sieving agent comprises a viscosity range of about 5 centipoise to about 15,000 centipoise when dissolved in water at 2 weight percent measured at about 25° C., or about 10 centipoise to about 10,000 centipoise as a 2% aqueous solutions measured at about 25° C., or about 15 centipoise to about 5,000 centipoise as a 2% aqueous solution measured at about 25° C. In some embodiments, a sieving agent comprises a viscosity average molecular weight of about 25 to about 1,500 kMv, or about 75-1,000 kMv, or about 85-800 kMv. In some embodiments, a nucleic acid amplification reaction mixture comprises a polymer at about 0.1 to about 20% weight per volume, or about 1-10% w/v, or about 2-5% w/v. For example, a sieving agent comprises an acrylamide polymer including polyacrylamide. In some embodiments, a sieving agent comprises a saccharide polymer. In some embodiments, a sieving agent comprises a polymer of glucose or galactose. In some embodiments, a saccharide polymers comprises cellulose, dextran, starch, glycogen, agar or agarose. In some embodiments, a sieving agent comprises sodium carboxymethyl 2-hydroxyethyl cellulose, sodium carboxymethyl cellulose, methyl cellulose, hydroxyl ethyl cellulose, 2-hydroxypropyl cellulose, carboxy methyl cellulose, hydroxyl propyl cellulose, hydroxyethyl methyl cellulose, hydroxybutyl methyl cellulose, (hydroxypropyl)methyl cellulose or hydroxyethyl ethyl cellulose. In some embodiments, a nucleic acid amplification reaction mixture comprises a mixture of different sieving agents, for example, a mixture of different cellulose derivatives.

In some embodiments, the present teachings provide methods for nucleic acid amplification, comprising at least one diffusion-reducing agent. In some embodiments, a diffusion-reducing agent comprises any compound that reduces diffusion/migration of polynucleotides from a region of higher concentration to one having a lower concentration. In some embodiments, a diffusion reducing agent comprises any compound that reduces migration of any component of a nucleic acid amplification reaction irrespective of size. In some embodiments, components of a nucleic acid amplification reaction include any one or any combination of particles attached with capture primers, polynucleotides, recombinase, polymerase, solution-phase primers, fusion primers nucleotides, divalent cations, ATP and/or co-factors. In some embodiments, a diffusion reducing agent comprises any compound that behaves as a crowding agent (U.S. Pat. No. 8,071,308 granted to Piepenburg). For example, a crowding agent can increase the concentration of one or more components in a nucleic acid amplification reaction by generating a crowded reaction environment. In some embodiments, a diffusion reducing agent comprises oligomers or polymers of ethylene oxide including polyethylene glycol (PEG), polyethylene oxides including triblock copolymers (e.g., Pluronics™), polystyrene, Ficoll, dextran, polyvinylpyrrolidone (PVP), or albumin.

In some embodiments, the disclosure relates generally to compositions, and related methods, systems, kits and apparatuses, comprising a single reaction mixture which can be used for a nucleic acid synthesis or nucleic acid amplification. The single reaction mixture can include any one or any combination of target polynucleotides, particles attached with capture primers, solution-phase primers, fusion primers, other additional primers, enzymes (e.g., polymerases), accessory proteins (e.g., recombinase, recombinase loading protein, single-stranded binding protein, helicase or topoisomerase), nucleotides, divalent cations, binding partners, co-factors and/or buffer. In some embodiments, the single reaction mixture contains an emulsion that provides compartmentalization for separately amplify target polynucleotides, or the single reaction mixture lacks an emulsion that provides compartmentalization. Optionally, the primers include any one or any combination of primers attached to a particle (e.g., immobilized capture primers) and/or soluble primers. Optionally, the enzymes comprise polymerases which include recombinant, fusion, mutant, heat-stable or heat labile forms. Optionally, the accessory proteins include any one or any combination of a single-stranded binding protein (e.g., SSB or gp32 protein), recombinase (e.g., recA or uvsX), recombinase loading protein (e.g., uvsY protein), helicase (e.g., uvsW protein), or topoisomerase. Optionally, the nucleotides can include compounds having structures the same as or similar to naturally-occurring nucleotides, or nucleotide analogs having derivatized base, sugar and/or phosphate groups, or labeled or non-labeled nucleotides. Optionally, the divalent cations include magnesium, manganese and/or calcium. Optionally, the binding partners include biotin and avidin-like compounds, such as avidin or streptavidin. Optionally, the buffer comprises a source of ions, such as KCl, K-acetate, NH₄-acetate, K-glutamate, NH₄Cl, or ammonium sulfate. Optionally, the buffer includes Tris, Tricine, HEPES, MOPS, ACES, MES, or inorganic buffers such as phosphate or acetate-based buffers which can provide a pH range of about 4-12. Optionally, the buffer includes chelating agents such as EDTA or EGTA. Optionally, the buffer includes dithiothreitol (DTT), glycerol, spermidine, and/or BSA (bovine serum albumin). Optionally, the buffer includes ATP.

In some embodiments, the nucleic acid amplification reaction includes a polymerase chain reaction (PCR) (U.S. Pat. Nos. 4,683,195 and 4,683,202 both granted to Mullis), ligase chain reaction (LCR) (Barany 1991 Proceedings National Academy of Science USA 88:189-193; Barnes 1994 Proceedings National Academy of Science USA 91:2216-2220), or isothermal self-sustained sequence reaction (Kwoh 1989 Proceedings National Academy of Science USA 86:1173-1177; WO 1988/10315; and U.S. Pat. Nos. 5,409,818, 5,399,491, and 5,194,370), or recombinase polymerase amplification (RPA) (U.S. Pat. No. 5,223,414 to Zarling, U.S. Pat. Nos. 5,273,881 and 5,670,316 both to Sena, and U.S. Pat. Nos. 7,270,981, 7,399,590, 7,435,561, 7,666,598, 7,763,427, 8,017,339, 8,030,000, 8,062,850, and 8,071,308).

PCR is a DNA synthesis reaction in which the reaction mixture is subjected to at least two complete reaction cycles, each reaction cycle comprising a denaturation period and at least one annealing and/or extension period, resulting if successful in synthesis of copies of a nucleic acid template in at least the initial cycles, and copies of the copies in at least the later cycles, generally resulting in geometric amplification of the template. In PCR, a pair of primers are provided that bind at each end of a target region, on opposite strands such that they each prime synthesis toward the other primer. The reaction is thermocycled so as to drive denaturation of the substrate in a high temperature step, annealing of the primers at a lower temperature step, and extension at a temperature which may be but is not necessarily higher than that of the annealing step. Geometric amplification occurs because the products of one cycle can serve as template in the next cycle.

LCR is a reaction in which at least a first probe and a second probe are provided. In an embodiment of LCR, the first and second probes are ligated in the presence of a template. The probes generally hybridize to the template to form a substrate for a ligase, i.e., a structure resembling nicked double-stranded DNA. A DNA ligase, such as a thermostable DNA ligase, is provided and product (ligated probes) is generated in a cyclic manner. A cycle of the reaction involves a denaturation step, an annealing step, and a ligation step. The reaction can provide geometric amplification, e.g., where probes are provided that hybridize to both strands of a double-stranded template, or where third and fourth probes complementary to the first and second probes are provided, such that the product from one cycle can function as a template in the next cycle.

An embodiment of isothermal self-sustained sequence reaction, also sometimes referred to as transcription-mediated amplification or TMA, involves synthesizing single-stranded RNA, single-stranded DNA and double-stranded DNA. The single-stranded RNA is a first template for a first primer, the single-stranded DNA is a second template for a second primer, and the double stranded DNA is a third template for synthesis of a plurality of copies of the first template. A sequence of the first primer or the second primer is complementary to a sequence of a target nucleic acid and a sequence of the first primer or the second primer is homologous to a sequence of the target nucleic acid. In an embodiment of an isothermal self-sustained sequence reaction, a first cDNA strand is synthesized by extension of the first primer along the target by an enzyme with RNA-dependent DNA polymerase activity, such as a reverse transcriptase. The first primer comprises a polymerase binding sequence (PBS) such as a PBS for a DNA-dependent RNA polymerase, such as T7, T3, or SP6 RNA polymerase. The first primer comprising a PBS is sometimes referred to as a promoter-primer. The first cDNA strand is rendered single-stranded, such as by denaturation or by degradation of the RNA, such as by an RNase H. The second primer then anneals to the first cDNA strand and is extended to form a second cDNA strand by an enzyme with DNA-dependent DNA polymerase activity, such as a reverse transcriptase or a DNA polymerase I. Forming the second cDNA strand renders the cDNA double-stranded, including the PBS. RNA can then be synthesized from the cDNA, which comprises the PBS, by a DNA-dependent RNA polymerase, such as T7, T3, or SP6 RNA polymerase, thereby providing a template for further events (extension of the first primer, rendering the product single-stranded, extension of the second primer, and RNA synthesis). Geometric amplification occurs because the RNA product can subsequently serve as a template and also because RNA products can be generated repeatedly from a cDNA comprising the PBS.

An embodiment of RPA can be performed isothermally and employs a recombinase to promote strand invasion of a double-stranded template by forward and reverse primers. The 3′ ends of the primers are extended, displacing template strands at least in part. Subsequent strand invasion/annealing events, including to previously produced extension products, occur and are followed by extension, resulting in amplification. In some embodiments, recombinase activity is supported by the presence of one or more recombinase accessory proteins, such as a recombinase loading protein and/or single-stranded binding protein.

In some embodiments, the disclosure relates generally to compositions, and related methods, systems, kits and apparatuses, comprising a nucleic acid synthesis or nucleic acid amplification reaction (amplification condition) that can be conducted under thermo-cycling or isothermal conditions, or a combination of both types of conditions. For example, the amplification condition can include alternating between thermocycling and isothermal amplification conditions, in any order.

In some embodiments thermo-cycling amplification conditions comprise a nucleic acid amplification reaction mixture that is subjected to an elevated temperature for a period of time that is sufficient to denature at least about 30-95% of the double-stranded target nucleic acids, and then subjected to a lower temperature for a period of time that is sufficient to permit hybridization between the single-stranded target nucleic acids and any of the primers (e.g., capture primer, reverse solution-phase primer, or fusion primer). In some embodiments, the increase and decrease temperature cycle is repeated at least once.

In some embodiments isothermal amplification conditions comprise a nucleic acid amplification reaction mixture that is subjected to a temperature variation which is constrained within a limited range during at least some portion of the amplification, including for example a temperature variation is within about 20° C., or about 10° C., or about 5° C., or about 1-5° C., or about 0.1-1° C., or less than about 0.1° C.

In some embodiments, an isothermal nucleic acid amplification reaction can be conducted for about 2, 5, 10, 15, 20, 30, 40, 50, 60 or 120 minutes, or longer. In some embodiments, an isothermal nucleic acid amplification reaction can be conducted for at least about 2 minutes. In some embodiments, an isothermal nucleic acid amplification reaction can be conducted for about 120 minutes or less. In some embodiments, an isothermal nucleic acid amplification reaction can be conducted for about 2 to about 120 minutes. In some embodiments, an isothermal nucleic acid amplification reaction can be conducted for about 2 to about 60 minutes. In some embodiments, an isothermal nucleic acid amplification reaction can be conducted for about 60 to about 120 minutes. In some embodiments, an isothermal nucleic acid amplification reaction can be conducted for about 2 to about 5 minutes. In some embodiments, an isothermal nucleic acid amplification reaction can be conducted for about 5 to about 10 minutes. In some embodiments, an isothermal nucleic acid amplification reaction can be conducted for about 10 to about 15 minutes. In some embodiments, an isothermal nucleic acid amplification reaction can be conducted for about 10 to about 15 minutes. In some embodiments, an isothermal nucleic acid amplification reaction can be conducted for about 10 to about 15 minutes. In some embodiments, an isothermal nucleic acid amplification reaction can be conducted for about 15 to about 20 minutes. In some embodiments, an isothermal nucleic acid amplification reaction can be conducted for about 20 to about 30 minutes. In some embodiments, an isothermal nucleic acid amplification reaction can be conducted for about 30 to about 40 minutes. In some embodiments, an isothermal nucleic acid amplification reaction can be conducted for about 40 to about 50 minutes. In some embodiments, an isothermal nucleic acid amplification reaction can be conducted for about 50 to about 60 minutes.

In some embodiments, an isothermal nucleic acid amplification reaction can be conducted at about 15-30° C., or about 30-45° C., or about 45-60° C., or about 60-75° C., or about 75-90° C., or about 90-93° C., or about 93-99° C.

In some embodiments, the disclosure relates generally to methods, and related compositions, systems, kits and apparatuses, that further include an enrichment step. In some embodiments, an amplified population of nucleic acids can include an affinity moiety. For example, in conducting any of the nucleic acid synthesis or amplification methods according to the present teachings, a solution-phase/reverse primer that is attached to an affinity moiety (e.g., biotin) can be used to conduct an amplification reaction to produce an amplified population of nucleic acids that are attached to the affinity moiety.

In some embodiments, an enrichment step comprises hybridizing a solution-phase enrichment primer comprising a binding partner or affinity moiety (e.g., biotin) for affinity-based enrichment of the amplified polynucleotides. The amplified polynucleotides may be attached to a particle when the enrichment primer is hybridized, and the particles comprising amplified polynucleotides that hybridize (such as through adaptors) to the enrichment primer may be separated from particles that do not comprising amplified polynucleotides that can hybridize to the enrichment primer.

In some embodiments, the enrichment step comprises forming a enrichment complex by binding the affinity moiety (which is attached to the amplified population of nucleic acids) with a purification particle (e.g., paramagnetic bead) that is attached to a receptor moiety (e.g., streptavidin). An example of purification particles include MyOne™ Beads from Dynabeads, which are paramagnetic beads attached to streptavidin. In some embodiments, a magnet can be used to separate/remove the enrichment complex from amplified population of nucleic acids that lack the affinity moiety. In some embodiments, the enrichment step can be repeated at least once. In some embodiment, the enrichment step is followed by one or more washing step.

In some embodiments, the disclosure relates generally to methods, and related compositions, systems, kits and apparatuses that further include at least one washing step. The washing step can be conducted at any time during the workflow for nucleic acid synthesis or amplification. In some embodiments, a washing step can remove excess or unreacted components of the nucleic acid synthesis (e.g., amplification) or enrichment reactions.

In some embodiments, any of the nucleic acid synthesis or amplification methods, or enrichment steps, according to the present teachings, can be conducted manually or by automation. In some embodiments, any one or any combination of the steps can be conducted manually or by automation, including: (1) forming a single reaction mixture, (2) conducting a nucleic acid amplification reaction, (3) enriching and/or (4) washing. For example, any reagents for a nucleic acid synthesis (e.g., amplification), enrichment or washing, can be deposited into, or removed from, a reaction vessel via manual or automated modes. In some embodiments, reagents for nucleic acid synthesis include any one or any combination of target polynucleotides, particles attached with capture primers, solution-phase primers, fusion primers, other additional primers, enzymes (e.g., polymerases), accessory proteins (e.g., recombinase, recombinase loading protein, single-stranded binding protein, helicase or topoisomerase), nucleotides, divalent cations, binding partners, co-factors and/or buffer.

In some embodiments, the disclosure relates generally to methods, and related compositions, systems, kits and apparatuses, which further include a digestion/cleaving reaction. For example, when a primer comprising at least one uridine as described above is used in an amplification reaction, a part of the primer can be digested. The digestion/cleaving can be a digestion that targets uridine. For example, one or more of UDG, fpg, or an appropriate exonuclease can be used to perform the digestion. The digestion reaction can also include a DNA polymerase, such as PolI, T4 PNK or Klenow. The digestion reaction can optionally include an antibody such as an anti-Taq antibody. In some embodiments, the digestion does not affect the annealing site for a sequencing and/or enrichment primer used in a subsequent sequencing and/or enrichment step.

In some embodiments, the disclosure relates generally to methods, and related compositions, systems, kits and apparatuses, which further include a sequencing reaction. In some embodiments, any target polynucleotide that has been amplified according to the present teachings can be sequenced. In some such embodiments, the present methods produce cleaner sequencing results by reducing, for example, the presence of primer dimers associated with the particles.

In some embodiments, any type of sequencing platform can be employed, including: sequencing by oligonucleotide probe ligation and detection (e.g., SOLiD′ from Life Technologies, WO 2006/084131), probe-anchor ligation sequencing (e.g., Complete Genomics™ or Polonator™), sequencing-by-synthesis (e.g., Genetic Analyzer and HiSeq™, from Illumina), pyrophosphate sequencing (e.g., Genome Sequencer FLX from 454 Life Sciences), ion-sensitive sequencing (e.g., Personal Genome Machine (PGM™) and Ion Proton™ Sequencer, both from Ion Torrent Systems, Inc.), and single molecule sequencing platforms (e.g., HeliScope™ from Helicos™).

In some embodiments, nucleic acids that have been synthesized, or have been amplified, according to the present teachings can be sequenced by any sequencing method, including sequencing-by-synthesis, ion-based sequencing involving the detection of sequencing byproducts using field effect transistors (e.g., FETs and ISFETs), chemical degradation sequencing, ligation-based sequencing, hybridization sequencing, pyrophosphate detection sequencing, capillary electrophoresis, gel electrophoresis, next-generation, massively parallel sequencing platforms, sequencing platforms that detect hydrogen ions or other sequencing by-products, and single molecule sequencing platforms. In some embodiments, a sequencing reaction can be conducted using at least one sequencing primer that can hybridize to any portion of the polynucleotide constructs, including a nucleic acid adaptor or a target polynucleotide.

In some embodiments, the disclosure relates generally to methods, as well as related systems, compositions, kits and apparatuses, for conducting a sequencing reaction on a support having one or more reaction sites coupled to a sensor.

In some embodiments, any target polynucleotide that is amplified according to the present teachings can be sequenced using methods that detect one or more byproducts of nucleotide incorporation. The detection of polymerase extension by detecting physicochemical byproducts of the extension reaction, can include pyrophosphate, hydrogen ion, charge transfer, heat, and the like, as disclosed, for example, in U.S. Pat. No. 7,948,015 to Rothberg et al.; and Rothberg et al, U.S. Patent Publication No. 2009/0026082, hereby incorporated by reference in their entireties. Other examples of methods of detecting polymerase-based extension can be found, for example, in Pourmand et al, Proc. Natl. Acad. Sci., 103: 6466-6470 (2006); Purushothaman et al., IEEE ISCAS, IV-169-172; Anderson et al, Sensors and Actuators B Chem., 129: 79-86 (2008); Sakata et al., Angew. Chem. 118:2283-2286 (2006); Esfandyapour et al., U.S. Patent Publication No. 2008/01666727; and Sakurai et al., Anal. Chem. 64: 1996-1997 (1992).

Reactions involving the generation and detection of ions are widely performed. The use of direct ion detection methods to monitor the progress of such reactions can simplify many current biological assays. For example, template-dependent nucleic acid synthesis by a polymerase can be monitored by detecting hydrogen ions that are generated as natural byproducts of nucleotide incorporations catalyzed by the polymerase. Ion-sensitive sequencing (also referred to as “pH-based” or “ion-based” nucleic acid sequencing) exploits the direct detection of ionic byproducts, such as hydrogen ions, that are produced as a byproduct of nucleotide incorporation. In one exemplary system for ion-based sequencing, the nucleic acid to be sequenced can be captured in a microwell, and nucleotides can be flowed across the well, one at a time, under nucleotide incorporation conditions. The polymerase incorporates the appropriate nucleotide into the growing strand, and the hydrogen ion that is released can change the pH in the solution, which can be detected by an ion sensor that is coupled with the well. This technique does not require labeling of the nucleotides or expensive optical components, and allows for far more rapid completion of sequencing runs. Examples of such ion-based nucleic acid sequencing methods and platforms include the Ion Torrent PGM™ or Proton™ sequencer (Ion Torrent™ Systems, Life Technologies Corporation).

In some embodiments, target polynucleotides produced using the methods, systems and kits of the present teachings can be used as a substrate for a biological or chemical reaction that is detected and/or monitored by a sensor including a field-effect transistor (FET). In various embodiments the FET is a chemFET or an ISFET. A “chemFET” or chemical field-effect transistor, is a type of field effect transistor that acts as a chemical sensor. It is the structural analog of a MOSFET transistor, where the charge on the gate electrode is applied by a chemical process. An “ISFET” or ion-sensitive field-effect transistor, is used for measuring ion concentrations in solution; when the ion concentration (such as H+) changes, the current through the transistor will change accordingly. A detailed theory of operation of an ISFET is given in “Thirty years of ISFETOLOGY: what happened in the past 30 years and what may happen in the next 30 years,” P. Bergveld, Sens. Actuators, 88 (2003), pp. 1-20.

In some embodiments, the FET may be a FET array. As used herein, an “array” is a planar arrangement of elements such as sensors or wells. The array may be one or two dimensional. A one dimensional array can be an array having one column (or row) of elements in the first dimension and a plurality of columns (or rows) in the second dimension. The number of columns (or rows) in the first and second dimensions may or may not be the same. The FET or array can comprise 10², 10³, 10⁴, 10⁵, 10⁶, 10⁷ or more FETs.

In some embodiments, one or more microfluidic structures can be fabricated above the FET sensor array to provide for containment and/or confinement of a biological or chemical reaction. For example, in one implementation, the microfluidic structure(s) can be configured as one or more wells (or microwells, or reaction chambers, or reaction wells, as the terms are used interchangeably herein) disposed above one or more sensors of the array, such that the one or more sensors over which a given well is disposed detect and measure analyte presence, level, and/or concentration in the given well. In some embodiments, there can be a 1:1 correspondence of FET sensors and reaction wells. Exemplary embodiments of FET sensor arrays can be found in U.S. Pat. Nos. 7,948,015; 8,262,900; 8,776,573; 8,208,712.

Microwells or reaction chambers are typically hollows or wells having well-defined shapes and volumes which can be manufactured into a substrate and can be fabricated using conventional microfabrication techniques, e.g. as disclosed in the following references: Doering and Nishi, Editors, Handbook of Semiconductor Manufacturing Technology, Second Edition (CRC Press, 2007); Saliterman, Fundamentals of BioMEMS and Medical Microdevices (SPIE Publications, 2006); Elwenspoek et al, Silicon Micromachining (Cambridge University Press, 2004); and the like. Examples of configurations (e.g. spacing, shape and volumes) of microwells or reaction chambers are disclosed in Rothberg et al, U.S. patent publication 2009/0127589; Rothberg et al, U.K. patent application GB24611127.

In some embodiments, the biological or chemical reaction can be performed in a solution or a reaction chamber that is in contact with, operatively coupled, or capacitively coupled to a FET such as a chemFET or an ISFET. The FET (or chemFET or ISFET) and/or reaction chamber can be an array of FETs or reaction chambers, respectively.

In some embodiments, a biological or chemical reaction can be carried out in a two-dimensional array of reaction chambers, wherein each reaction chamber can be coupled to a FET, and each reaction chamber is no greater than 10 μm³ (i.e., 1 pL) in volume. In some embodiments each reaction chamber is no greater than 0.34 pL, 0.096 pL or even 0.012 pL in volume. A reaction chamber can optionally be no greater than 2, 5, 10, 15, 22, 32, 42, 52, 62, 72, 82, 92, or 102 square microns in cross-sectional area at the top. Preferably, the array has at least 10², 10³, 10⁴, 10⁵, 10⁶, 10⁷, 10⁸, 10⁹, or more reaction chambers. In some embodiments, at least one of the reaction chambers is operatively coupled to at least one of the FETs.

FET arrays as used in various embodiments according to the disclosure can be fabricated according to conventional CMOS fabrications techniques, as well as modified CMOS fabrication techniques and other semiconductor fabrication techniques beyond those conventionally employed in CMOS fabrication. Additionally, various lithography techniques can be employed as part of an array fabrication process.

Exemplary FET arrays suitable for use in the disclosed methods, as well as microwells and attendant fluidics, and methods for manufacturing them, are disclosed, for example, in U.S. Patent Publication No. 20100301398; U.S. Patent Publication No. 20100300895; U.S. Patent Publication No. 20100300559; U.S. Patent Publication No. 20100197507, U.S. Patent Publication No. 20100137143; U.S. Patent Publication No. 20090127589; and U.S. Patent Publication No. 20090026082, which are incorporated by reference in their entireties.

In one aspect, the disclosed methods, compositions, systems, apparatuses and kits can be used for carrying out label-free nucleic acid sequencing, and in particular, ion-based nucleic acid sequencing. The concept of label-free detection of nucleotide incorporation has been described in the literature, including the following references that are incorporated by reference: Rothberg et al, U.S. patent publication 2009/0026082; Anderson et al, Sensors and Actuators B Chem., 129: 79-86 (2008); and Pourmand et al, Proc. Natl. Acad. Sci., 103: 6466-6470 (2006). Briefly, in nucleic acid sequencing applications, nucleotide incorporations are determined by measuring natural byproducts of polymerase-catalyzed extension reactions, including hydrogen ions, polyphosphates, PPi, and Pi (e.g., in the presence of pyrophosphatase). Examples of such ion-based nucleic acid sequencing methods and platforms include the Ion Torrent PGM™ or Proton™ sequencer (Ion Torrent™ Systems, Life Technologies Corporation).

In some embodiments, the disclosure relates generally to methods for sequencing nucleic acids that have been amplified by the teachings provided herein. In one exemplary embodiment, the disclosure relates generally to a method for obtaining sequence information from polynucleotides, comprising: (a) amplifying target polynucleotides; and (b) sequencing the amplified target polynucleotides by performing template-dependent nucleic acid synthesis using at least one of the amplified target polynucleotides produced during step (a) as a template. The amplifying can optionally be performed according to any of the amplification methods described herein.

In some embodiments, the template-dependent synthesis includes incorporating one or more nucleotides in a template-dependent fashion into a newly synthesized nucleic acid strand.

Optionally, the methods can further include producing one or more ionic byproducts of such nucleotide incorporation.

In some embodiments, the methods can further include detecting the incorporation of the one or more nucleotides into the sequencing primer. Optionally, the detecting can include detecting the release of hydrogen ions.

In another embodiment, the disclosure relates generally to a method for sequencing a nucleic acid, comprising: (a) amplifying target polynucleotides to generate at least one particle attached with a polynucleotide population containing a portion of one of the target polynucleotides, according to the teachings disclosed herein; and (b) disposing the particles into a reaction chambers, wherein one or more of the reaction chambers are in contact with a field effect transistor (FET). Optionally, the method further includes contacting the amplified nucleic acids which are disposed into one of the reaction chambers, with a polymerase thereby synthesizing a new nucleic acid strand by sequentially incorporating one or more nucleotides into a nucleic acid molecule. Optionally, the method further includes generating one or more hydrogen ions as a byproduct of such nucleotide incorporation. Optionally, the method further includes detecting the incorporation of the one or more nucleotides by detecting the generation of the one or more hydrogen ions using the FET.

In some embodiments, the detecting includes detecting a change in voltage and/or current at the at least one FET within the array in response to the generation of the one or more hydrogen ions.

In some embodiments, the FET can be selected from the group consisting of: ion-sensitive FET (isFET) and chemically-sensitive FET (chemFET).

In some embodiments, the disclosure relates generally to methods (and related compositions, systems, kits and apparatuses) for nucleic acid sequencing, comprising identifying a series of contiguous nucleotides in a nucleic acid template according to any of the methods disclosed herein.

One exemplary system involving sequencing via detection of ionic byproducts of nucleotide incorporation is the Ion Torrent PGM™ or Proton™ sequencer (Life Technologies), which is an ion-based sequencing system that sequences nucleic acid templates by detecting hydrogen ions produced as a byproduct of nucleotide incorporation. Typically, hydrogen ions are released as byproducts of nucleotide incorporations occurring during template-dependent nucleic acid synthesis by a polymerase. The Ion Torrent PGM™ or Proton™ sequencer detects the nucleotide incorporations by detecting the hydrogen ion byproducts of the nucleotide incorporations. The Ion Torrent PGM™ or Proton™ sequencer can include a plurality of nucleic acid templates to be sequenced, each template disposed within a respective sequencing reaction well in an array. The wells of the array can each be coupled to at least one ion sensor that can detect the release of H⁺ ions or changes in solution pH produced as a byproduct of nucleotide incorporation. The ion sensor comprises a field effect transistor (FET) coupled to an ion-sensitive detection layer that can sense the presence of H⁺ ions or changes in solution pH. The ion sensor can provide output signals indicative of nucleotide incorporation which can be represented as voltage changes whose magnitude correlates with the H⁺ ion concentration in a respective well or reaction chamber. Different nucleotide types can be flowed serially into the reaction chamber, and can be incorporated by the polymerase into an extending primer (or polymerization site) in an order determined by the sequence of the template. Each nucleotide incorporation can be accompanied by the release of H⁺ ions in the reaction well, along with a concomitant change in the localized pH. The release of H⁺ ions can be registered by the FET of the sensor, which produces signals indicating the occurrence of the nucleotide incorporation. Nucleotides that are not incorporated during a particular nucleotide flow may not produce signals. The amplitude of the signals from the FET can also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers can permit the instrument to resolve the sequence of many nucleic acid templates simultaneously. Further details regarding the compositions, design and operation of the Ion Torrent PGM™ or Proton™ sequencer can be found, for example, in U.S. patent application Ser. No. 12/002,781, now published as U.S. Patent Publication No. 2009/0026082; U.S. patent application Ser. No. 12/474,897, now published as U.S. Patent Publication No. 2010/0137143; and U.S. patent application Ser. No. 12/492,844, now published as U.S. Patent Publication No. 2010/0282617, all of which applications are incorporated by reference herein in their entireties.

In a typical embodiment of ion-based nucleic acid sequencing, nucleotide incorporations can be detected by detecting the presence and/or concentration of hydrogen ions generated by polymerase-catalyzed extension reactions. In one embodiment, templates, optionally pre-bound to a sequencing primer and/or a polymerase, can be loaded into reaction chambers (such as the microwells disclosed in Rothberg et al, cited herein), after which repeated cycles of nucleotide addition and washing can be carried out. In some embodiments, such templates can be attached as clonal populations to a solid support, such as particles, bead, or the like, and said clonal populations are loaded into reaction chambers.

In another embodiment, the templates, optionally bound to a polymerase, are distributed, deposited or positioned to different sites of the array. The sites of the array include primers and the methods can include hybridizing different templates to the primers within different sites.

In each addition step of the cycle, the polymerase can extend the primer by incorporating added nucleotide only if the next base in the template is the complement of the added nucleotide. If there is one complementary base, there is one incorporation, if two, there are two incorporations, if three, there are three incorporations, and so on. With each such incorporation there is a hydrogen ion released, and collectively a population of templates releasing hydrogen ions changes the local pH of the reaction chamber. The production of hydrogen ions is monotonically related to the number of contiguous complementary bases in the template (as well as the total number of template molecules with primer and polymerase that participate in an extension reaction). Thus, when there are a number of contiguous identical complementary bases in the template (i.e. a homopolymer region), the number of hydrogen ions generated, and therefore the magnitude of the local pH change, can be proportional to the number of contiguous identical complementary bases. If the next base in the template is not complementary to the added nucleotide, then no incorporation occurs and no hydrogen ion is released. In some embodiments, after each step of adding a nucleotide, an additional step can be performed, in which an unbuffered wash solution at a predetermined pH is used to remove the nucleotide of the previous step in order to prevent misincorporations in later cycles. In some embodiments, the after each step of adding a nucleotide, an additional step can be performed wherein the reaction chambers are treated with a nucleotide-destroying agent, such as apyrase, to eliminate any residual nucleotides remaining in the chamber, which may result in spurious extensions in subsequent cycles.

In one exemplary embodiment, different kinds of nucleotides are added sequentially to the reaction chambers, so that each reaction can be exposed to the different nucleotides one at a time. For example, nucleotides can be added in the following sequence: dATP, dCTP, dGTP, dTTP, dATP, dCTP, dGTP, dTTP, and so on; with each exposure followed by a wash step. The cycles may be repeated for 50 times, 100 times, 200 times, 300 times, 400 times, 500 times, 750 times, or more, depending on the length of sequence information desired.

In some embodiments, sequencing can be performed according to the user protocols supplied with the PGM™ or Proton™ sequencer. Protocols for ion-based sequencing using the Ion Torrent PGM™ sequencer are available from Thermo Fisher Scientific.

In some embodiments, the disclosure relates generally to methods for sequencing a population of template polynucleotides, comprising: (a) generating a plurality of amplicons by clonally amplifying a plurality of target polynucleotides onto a plurality of particles, wherein the amplifying is performed within a single continuous phase of a reaction mixture and wherein at least 10%, 20%, 30%, 40%, 50%, 60%, 70%, 80%, 90%, or 95% of the resulting amplicons are substantially monoclonal in nature. In some embodiments, a sufficient number of substantially monoclonal amplicons are produced in a single amplification reaction to generate at least 100 MB, 200 MB, 300 MB, 400 MB, 500 MB, 750 MB, 1 GB or 2 GB of AQ20 sequencing reads on an Ion Torrent PGM™ 314, 316 or 318 sequencer. The term “AQ20 and its variants, as used herein, refers to a particular method of measuring sequencing accuracy in the Ion Torrent PGM™ sequencer. Accuracy can be measured in terms of the Phred-like Q score, which measures accuracy on logarithmic scale that: Q10=90%, Q20=99%, Q30=99.9%, Q40=99.99%, and Q50=99.999%. For example, in a particular sequencing reaction, accuracy metrics can be calculated either through prediction algorithms or through actual alignment to a known reference genome. Predicted quality scores (“Q scores”) can be derived from algorithms that look at the inherent properties of the input signal and make fairly accurate estimates regarding if a given single base included in the sequencing “read” will align. In some embodiments, such predicted quality scores can be useful to filter and remove lower quality reads prior to downstream alignment. In some embodiments, the accuracy can be reported in terms of a Phred-like Q score that measures accuracy on logarithmic scale such that: Q10=90%, Q17=98%, Q20=99%, Q30=99.9%, Q40=99.99%, and Q50=99.999%. In some embodiments, the data obtained from a given polymerase reaction can be filtered to measure only polymerase reads measuring “N” nucleotides or longer and having a Q score that passes a certain threshold, e.g., Q10, Q17, Q100 (referred to herein as the “NQ17” score). For example, the 100Q20 score can indicate the number of reads obtained from a given reaction that are at least 100 nucleotides in length and have Q scores of Q20 (99%) or greater. Similarly, the 200Q20 score can indicate the number of reads that are at least 200 nucleotides in length and have Q scores of Q20 (99%) or greater.

In some embodiments, the accuracy can also be calculated based on proper alignment using a reference genomic sequence, referred to herein as the “raw” accuracy. This is single pass accuracy, involving measurement of the “true” per base error associated with a single read, as opposed to consensus accuracy, which measures the error rate from the consensus sequence which is the result of multiple reads. Raw accuracy measurements can be reported in terms of “AQ” scores (for aligned quality). In some embodiments, the data obtained from a given polymerase reaction can be filtered to measure only polymerase reads measuring “N” nucleotides or longer having a AQ score that passes a certain threshold, e.g., AQ10, AQ17, AQ100 (referred to herein as the “NAQ17” score). For example, the 100AQ20 score can indicate the number of reads obtained from a given polymerase reaction that are at least 100 nucleotides in length and have AQ scores of AQ20 (99%) or greater. Similarly, the 200AQ20 score can indicate the number of reads that are at least 200 nucleotides in length and have AQ scores of AQ20 (99%) or greater.

In some embodiments, the disclosure relates generally to methods, and related compositions, systems, kits and apparatuses, that further comprise depositing one or more particles onto a surface such as a detection area of a nucleic acid sequencing instrument or a location where nucleic acid sequencing reactions occur, wherein at least one particle is prepared according to the present teachings and is attached with a polynucleotide population containing a portion of one of the target polynucleotides. In some embodiments, the polynucleotide population is a substantially monoclonal polynucleotide population.

In some embodiments, the detection area of a nucleic acid sequencing instrument or the location where nucleic acid sequencing reactions occur includes a planar surface, flowcell, channel, reaction chamber, or an array of reaction chambers.

In some embodiments, reagents can be delivered to the detection area of a nucleic acid sequencing instrument, or delivered to the location where nucleic acid sequencing reactions occur, to conduct a nucleic acid sequencing reaction.

In some embodiments, nucleic acid sequencing reactions can be conducted by flowing sequencing reagents onto a planar surface, flowcell, channel, reaction chamber, or an array of reaction chambers having the deposited particles which are attached with a polynucleotide population containing a portion of one of the target polynucleotides.

In some embodiments, sequencing reagents can include any combination of polymerases, nucleotides, cations, and/or buffers. In some embodiments, sequencing reactions include: chemical degradation sequencing reactions; enzyme cascade reactions; chain-termination sequencing reactions; sequencing-by-synthesis reactions; sequencing-by-ligation reactions; and ion-detection-based reactions.

In some embodiments, the particles can be deposited into reaction chambers organized in an array, where the reaction chambers are coupled with at least one ion-sensitive sensor. Typically, a reaction chamber can hold one particle (e.g., which is attached with a population of polynucleotides). In some embodiments, a reaction chamber can also hold particles conjugated with enzymes (e.g., enzyme-beads). The enzyme-beads can be microparticles coated with one or more types of enzymes for conducting an enzyme cascade reaction. The enzyme cascade can be a pyrophosphate-sulfurylase-luciferase enzyme cascade.

In some embodiments, a sequencing reaction comprises: sequencing by oligonucleotide probe ligation and detection (e.g., SOLiD™), probe-anchor ligation sequencing (e.g., Complete Genomics or Polonator™), sequence-by-synthesis (e.g., Illumina), pyrophosphate sequencing (e.g., 454 Life Sciences), ion-sensitive sequencing (e.g., Personal Genome Machine (PGM™) and Ion Proton™ Sequencer, both from Ion Torrent Systems, Inc.) and single molecule sequencing platforms (e.g., Helicos™).

In some embodiments, the disclosure relates generally to compositions, as well as related systems, methods, kits and apparatuses, comprising a single reaction mixture that contains an emulsion to provide compartmentalization for separately amplifying different target polynucleotides. In some embodiments, the emulsion comprises two immiscible liquid phases. In some embodiments, two immiscible liquid phases are mixed together to make the emulsion. In some embodiments, one of the liquid phases is dispersed in the other. Optionally, the emulsion comprises a discontinuous hydrophilic phase and a continuous hydrophobic phase. Optionally, the discontinuous hydrophilic phase is surrounded by the continuous hydrophobic phase. Optionally, the emulsion comprises at least one hydrophilic phase compartment (e.g., droplet or micro-reactor) surrounded by a continuous hydrophobic phase. Optionally, the discontinuous hydrophilic phase provides a compartment. Optionally, the emulsion comprises a plurality of hydrophilic phase droplets and a continuous hydrophobic phase. Optionally, the emulsion comprises a plurality of aqueous droplets and a continuous hydrophobic phase. Optionally, the emulsion comprises at least one aqueous droplet. Optionally, the at least one aqueous droplet includes one or more particles. Optionally, the at least one aqueous droplet includes one or more different target polynucleotides. Optionally, the emulsion includes at least one aqueous droplet that includes one or more particles of the first type, the second type, or particles of both the first and second type. Optionally, the emulsion includes at least one aqueous droplet that includes one or more different target polynucleotides.

Optionally, the emulsion comprises a mixture of an aqueous liquid and a water-immiscible organic liquid. Optionally, the emulsion comprises at least one anionic, cationic or non-ionic surfactant. Optionally, the emulsion can have a droplet-type dispersion comprising oil-in-water, water-in-oil, or a bicontinuous microemulsion.

In some embodiments, the water immiscible organic liquid comprises an oil. In some embodiments, the oil can be from a natural source, including animal (e.g., tallow or lard), fish (e.g., fish oil), shark, seeds, nuts or plants (e.g., vegetable oils). Optionally, the oil can be from derived from petroleum, including mineral oils. Optionally, the oil comprises a fluorochemical oil, polyalphaolefin or ester oil.

In some embodiments, the surfactant includes small molecule surfactants, polymeric surfactants, triblock co-polymer surfactants or non-ionic block copolymer surfactants. Optionally, the surfactant comprises a sorbitan oleate or a silicone surfactant.

Optionally, the hydrophilic phase compartment can contain at least two different types of particles. Optionally, the hydrophilic phase compartment can contain at least two different types of target polynucleotides.

### EXAMPLES

The following examples are provided to illustrate certain disclosed embodiments and are not to be construed as limiting the scope of this disclosure in any way.

### Example 1. Method 1: Sequential Primer Sequencing Approach Using a Truncated Primer

Methodologies were investigated to incorporate the sequential primer sequencing approach that were compatible with current libraries using multiple distinct primers.

The first approach using current libraries is termed “Method 1” and is outlined in FIG. 3. This approach used a standard library in which two adaptors, B and A1, flanked 200 bp of insert sequence as shown in Step 1. The A1 adaptor comprised insert-proximal and insert-distal portions that can be bound by different primers. The templating method was RPA (22 pellets from TwistAmp® basic kit from TwistDx). This approach also used beads attached with B-adaptor capture primers.

The B adaptor sequence in the library was 5′-CCTATCCCCTGTGTGCCTTGGCAGTCTCAG-3′ (top strand) (SEQ ID NO: 1). The A1 adaptor sequence was 5′-CCATCTCATC CCTGCGTGTC TCCGACTCAG-3′ (SEQ ID NO: 2).

An RPA amplification was performed using a bead-attached capture primer (sequence CCACTACGCC TCCGCTTTCC TCTCTATG-3′) (SEQ ID NO: 3) comprising sequence complementary to adaptor B and a solution-phase primer, Seq²_21 mer (sequence GCAGTCTCAG TCCATCTCAT C-3′) (SEQ ID NO: 4) (corresponding to Tail_A2 in FIG. 3), comprising a 3′ segment complementary to the insert-distal end of the A1 adaptor and a 5′ segment (tail) not complementary to the A1 adaptor, as shown in Step 2. In a control reaction representing the “standard approach”, a primer whose sequence was CCATCTATCC CTGCGTGTCT CCGACTCAG (SEQ ID NO: 5) (“T_PCR_A”) was used instead of Seq²_21 mer. The reactions were incubated for 25 minutes. The amplifications using Seq²_21 mer and T_PCR_A generated library beads. Primer dimer beads lacking insert sequence were also formed.

The reactions were stopped using 100 mM EDTA, centrifugation, and then using 1% SDS. The beads were again centrifuged and the solution was replaced with annealing (PBST) buffer. The beads were again centrifuged and volume was adjusted to 100 uL by removing excess buffer.

A mixture of biotinylated (20%)/non-biotinylated (80%) A17mer primer (sequence CCCTGCGTGT CTCCGAC) (SEQ ID NO: 6) was added, followed by vortexing. Tubes were heated to 95° C. for 5 min, then 37° C. for 5 min. Excess A17mer was removed by washing. A standard enrichment process was performed.

Ion torrent sequencing was performed using the Ion Proton 1 platform and a standard sequencing primer (sequence: 5′-CCATCTCATCCCTGCGTGTCTCCGAC-3′ (SEQ ID NO: 7). Alternatively, the A17mer primer can be used as the sequencing primer.

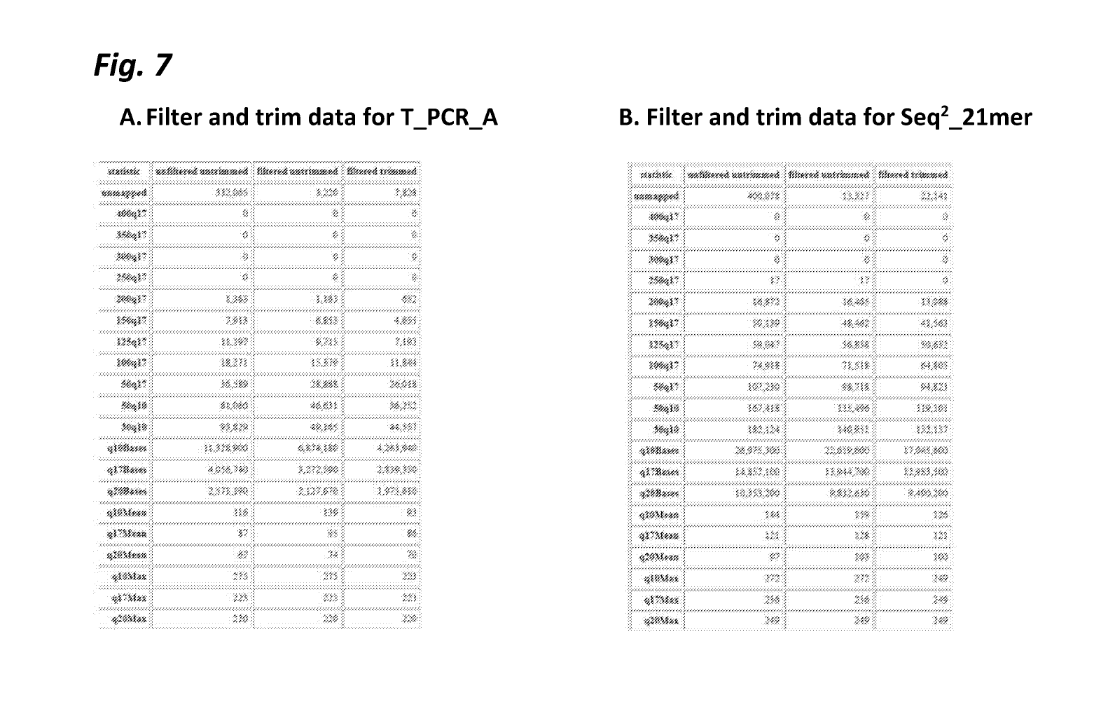

Results are shown for the T_PCR_A (FIG. 7A) versus the Seq²_21 mer (FIG. 7B) reactions. Using the Seq²_21 mer primer, the reads lost to filter and trim decreased to 12.61% compared to 30.00% for the T_PCR_A primer, indicating cleaner beads and fewer low-quality beads. Additionally, the Signal/Noise Ratio for the Seq²_21 mer experiment was 1.93, increased from 1.67 for the T_PCR_A experiment. The primer dimer (i.e., adaptor dimer) rate was reduced from 1.88% with the standard T_PCR_A method to 0.12% with the Seq²_21 mer method.

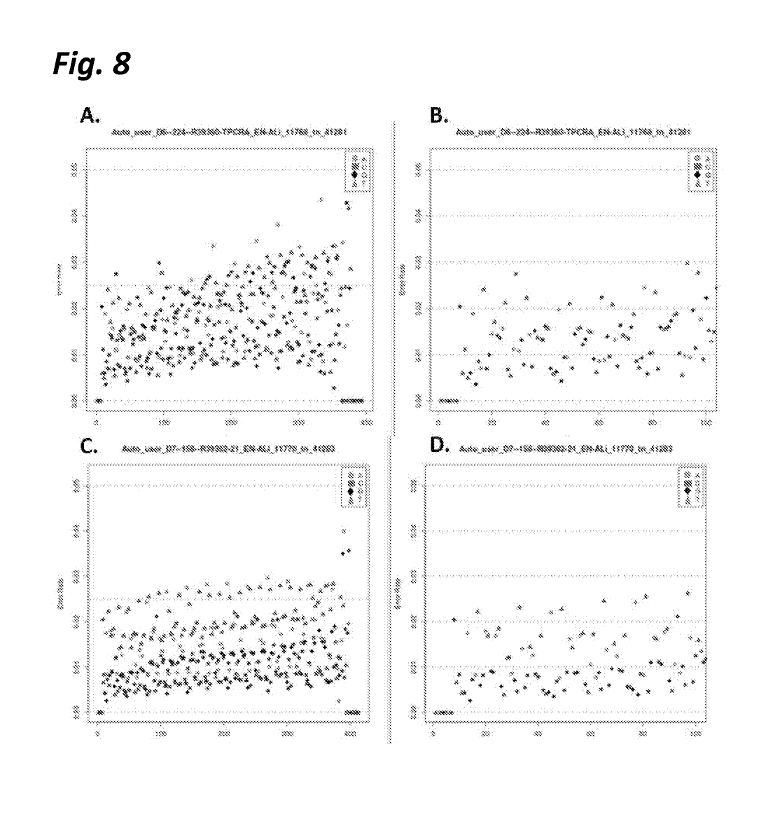

FIGS. 8A-8D show the error rates per cycle for T_PCR_A (FIGS. 8A and 8B) and for Seq²_21 mer (FIGS. 8C and 8D). FIGS. 8A and 8C show error rates of each base at each flow of nucleotides over 400 nucleotide flows, while FIGS. 9B and 9D show error rates over 100 nucleotide flows. These data on error rate indicate that the data using the Seq²_21 mer approach have a more uniform error rate compared with the standard T_PCR_A approach. This may indicate a cleaner bead system is produced using Seq² approach.

Barcoded libraries were prepared using either barcode 11 or 12, each including a 70 bp insert sequence flanked by B and A adaptor sequences. Bead templating reactions were conducted in manner similar to the protocol described above, using a non-emulsion, recombinase polymerase amplification (RPA) reaction, with 1 billion templating beads carrying B-capture primers, a barcoded library, and solution-phase amplification primers (500 nM). Solution-phase amplification primers included either the standard A adaptor (C for Control), or tailed primers having standard A and an A2 tail (T for Tailed)(e.g., see FIG. 3). Templated beads were hybridized with enrichment primers that hybridized to the standard A adaptor sequence. About 50% of the enrichment primers were biotinylated at their 5′ ends. Two different hybridization conditions were tested: 50° C. (H for high temperature) and 37° C. (L for low temperature). The enriched templated beads were analyzed by Guava flow cytometry to examine the yield of beads templated with primer-dimers or library molecules, and templated beads were sequenced on an Ion Torrent sequencing chip using the standard sequencing primer that hybridizes to the standard A adaptor sequence. The samples were spiked, or not spiked, with non-templated beads (dud beads) which are used as an internal reference for yield. Flow cytometry analysis of the templated beads after enrichment for under low (L) or high (H) hybridization conditions. shows beads templated in the RPA reaction with the standard A primers (C) and carrying barcoded library 11 or 12, yield decreased enrichment when high temperature (H) hybridization conditions are used compared to low temperature (L) hybridization conditions (i.e., standard, previously described method). In contrast, however, yield of enriched beads is not significantly different under low (L) or high (H) temperature hybridization conditions using methods of the present invention. More importantly, a comparison of the quality of the sequencing data obtained from the two barcoded libraries indicates that the signal-to-noise improves when the tailed primer was used in the bead templating/enrichment reactions. For example, the SNR of the 11TH sample is 13.2 compared to the 12CH which has an SNR of 9.4 and compared to 11CH which has an SNR of 8.2. Additionally, the AQ17mean of the 11TH sample is 27.8, compared to the 12CH sample which has an AQ17mean of 19.2 and compared to 11CH which has an AQ17mean of 18.2. Table 1 lists sequencing quality metrics obtained from sequencing data obtained from the two barcoded libraries on four different sequencing chips:

TABLE 1
Sequencing metrics

| | | Guava | read | bad | | key | key | rel. | AQ17 | Q17 | mQ20 | AQ20 | HP1 | HP2 |
|---|---|---|---|---|---|---|---|---|---|---|---|---|---|---|
| | Barcode | count | count | key | filtered | peak | snr | loading % | mean | mean | Alignment | Mean | acc % | acc % |
| Chip 1 | 11TH | 7.2E+07 | 6091 | 317 | 939 | 66.26 | 13.17 | 70.65 | 27.76 | 72 | 2364 | 37 | 98.49 | 95.94 |
| | 12CH | 1.1E+08 | 2041 | 3253 | 3711 | 45.73 | 9.38 | 23.67 | 19.23 | 71 | 424 | 30 | 97.87 | 95.71 |
| Chip 2 | 11TL | 7.4E+07 | 7608 | 335 | 1044 | 64.91 | 13.09 | 68.73 | 30.7 | 79 | 3206 | 41 | 98.46 | 96.18 |
| | 12CL | 1.3E+08 | 2959 | 3323 | 3877 | 44.4 | 9.14 | 26.73 | 21.53 | 71 | 715 | 34 | 97.97 | 95.86 |
| Chip 3 | 12TH | 1.8E+07 | 3852 | 820 | 1223 | 60.37 | 11.43 | 86.08 | 29.36 | 77 | 1589 | 39 | 98.4 | 96.19 |
| | 11CH | 5.8E+07 | 136 | 1676 | 1782 | 44.31 | 8.24 | 3.04 | 18.23 | 66 | 26 | 33 | 99 | 95.87 |
| Chip 4 | 12TL | 5.6E+07 | 10995 | 635 | 1336 | 65.09 | 13.54 | 89.06 | 33.24 | 85 | 5138 | 42 | 98.62 | 96.67 |
| | 11CL | 7.7E+07 | 854 | 1908 | 2319 | 44.61 | 9.61 | 6.92 | 18.65 | 67 | 157 | 32 | 98.13 | 95.99 |

Many quality metrics improve when the tailed primer is used in the bead templating/enrichment reaction.

### Example 2. Method 2: Sequential Primer Sequencing Approach Using Library with Fusion Adaptors

A second approach, termed Method 2, is outlined in FIG. 4. A tailed primer is not needed for this method. Method 2 uses a library generated with two adaptors flanking an insert—the A1-A2 fusion adaptor and the B adaptor—as shown in Step 1.

A solution phase primer, A2′, and a bead-attached capture primer are used for amplification, as shown in Step 2. The A2′ primer is complementary to the A2 portion of the A1-A2 fusion adaptor and does not comprise sequence complementary to the A1 portion of the A1-A2 fusion adaptor. The capture primer comprises sequence complementary to the B adaptor. The amplification generates library beads. Primer dimer beads lacking insert sequence may also result from the amplification reaction. The primer dimer beads also lack A1 sequence.

The library beads are then sequenced using primer A1′, as shown in Step 3. Primer A1′ is complementary to the A1 portion of the A1-A2 fusion adaptor. Primer dimer beads lack the A1 adaptor, so primer A1′ is selective for library beads over primer dimer beads. In the case of library plus primer dimer mixed beads, primer A1′ is selective for sequencing of the library insert over the primer dimer.

### Example 3: Sequential Primer Sequencing Approach Using Library with Overlapping and Truncated Domains

In yet another related approach, amplification and enrichment domains may comprise overlapping sequences, though less than 100% as in standard methods, but to reduce the size of the adaptor. The library molecules contained an insert sequence flanked by an A and B adaptor. The A adaptor sequence included overlapping amplification and enrichment domains.

A adaptor:
(SEQ ID NO: 8)
CCATCTCATCCCTGCGTGTCTCCGACTCAG.
Amplification primer:
(SEQ ID NO: 9)
CCATCTCATCCCTGCGTGTC.
Enrichment primer:
(SEQ ID NO: 10)
CATCCCTGCGTGTCTCCGAC

A bead templating reaction was conducted using a non-emulsion, recombinase polymerase amplification (RPA) reaction, with templating beads carrying B-capture primers, the library molecules, and solution-phase amplification primers (SEQ ID NO:9), where approximately 4% of the soluble amplification primers were biotinylated at their 5′ ends. The RPA reaction was set up using dehydrated pellets from a TWISTAMP basic kit from TwistDx. The RPA reaction was incubated at 40° C. for 20 minutes, and then heat-killed at 68° C. for 10 minutes.

The templated beads were reacted with MYONE Streptavidin Cl beads (Thermo Fisher Scientific), and the captured templated beads were enriched using a magnet. The melt-off step was conducted with 100 uL of 50 mM NaOH. The recovered templated beads were hybridized with enrichment primers (SEQ ID NO:10), where about 10% of the enrichment primers were biotinylated at their 5′ ends. The melt-off solution was neutralized by adding 20 uL of 1M Tris-HCl (pH 7). 5 uL of 10× Annealing buffer was added. The hybridization reaction was heated to 95° C. for 2 minutes, then incubated at 37° C. for 2 minutes. 150 uL of MYONE Streptavidin Cl beads was added, and incubated at room temperature for 5 minutes, and the captured templated beads were enriched with a magnet. The templated beads were recovered from the MYONE beads using a NaOH melt-off procedure. The recovered beads were washed. The library molecules on the templated beads were analyzed by Guava flow cytometry, and were sequenced on an Ion Torrent sequencing chip using the standard Ion Torrent sequencing primer or the enrichment primer (SEQ ID NO:10).

Guava flow cytometry data of pre-enriched and post-enriched templated beads demonstrated, approximately 387 million pre-enriched templated beads were recovered that contained about 23% primer-dimer beads, and approximately 315 million post-enriched templated beads were recovered that contained about 12% primer-dimer beads.

In additional approaches, bead templating and enrichment steps can be performed using truncated versions of amplification and enrichment primers to reduce overlapping sequence regions within the A adaptor sequenceas follows: truncated amplification primer: CCATCTCATCCCTGCGT (SEQ ID NO:11); truncated enrichment primer: CCCTGCGTGTCTCCGAC (SEQ ID NO:12).

1.-108. (canceled)

109. A method of producing at least one particle comprising a template sequence, comprising:
a) forming a reaction mixture by contacting:
(i) a template polynucleotide, wherein the template polynucleotide comprises, 5′ to 3′, a second adaptor sequence, a template sequence, and a first adaptor sequence,
(ii) at least one particle having a plurality of a capture primer attached thereon, wherein the capture primer is capable of hybridizing to the first adaptor sequence, and
(iii) a plurality of a solution-phase primer, wherein the solution phase primer comprises, 5′ to 3′, a fourth adaptor sequence and a third adaptor sequence, wherein the third adaptor sequence is capable of hybridizing to the complement of the second adaptor sequence;
b) subjecting the reaction mixture to a nucleic acid amplification condition, thereby generating at least one particle attached to a polynucleotide population containing at least one polynucleotide that comprises, 5′ to 3′, the capture primer sequence, the complement of the template sequence, the complement of the third adaptor sequence, and the complement of the fourth adaptor sequence.

110. A method of producing at least a set of particles, wherein a first set of particles comprises a first template sequence and an optional second set of particles comprises a second template sequence, wherein the first and second template sequences may be the same or different, comprising:
a) forming a reaction mixture by contacting:
(i) a first template polynucleotide, wherein the first template polynucleotide comprises, 5′ to 3′, a second adaptor sequence, a first identifier sequence, the first template sequence, and a first adaptor sequence,
(ii) an optional second template polynucleotide, wherein the second template polynucleotide comprises, 5′ to 3′, a second adaptor sequence, a second identifier sequence, the second template sequence, and a third adaptor sequence,
(iii) a first set of particles having a plurality of a first capture primer attached thereon, wherein the first capture primer is capable of hybridizing to the first adaptor sequence,
(iv) an optional second set of particles having a plurality of a second capture primer attached thereon, wherein the second capture primer is capable of hybridizing to the third adaptor sequence,
(v) a plurality of a solution-phase primer, wherein the solution phase primer comprises, 5′ to 3′, a fourth adaptor sequence and a fifth adaptor sequence, wherein the fifth adaptor sequence is capable of hybridizing to the complement of the second adaptor sequence;
b) subjecting the reaction mixture to a nucleic acid amplification condition, thereby generating:
(i) a first set of particles attached to a first polynucleotide population containing at least one polynucleotide that comprises, 5′ to 3′, the first capture primer sequence, the complement of the first template sequence, the complement of the first identifier sequence, the complement of the fifth adaptor sequence, and the complement of the fourth adaptor sequence; and (ii) an optional second set of particles attached to a second polynucleotide population containing at least one polynucleotide that comprises, 5′ to 3′, the second capture primer sequence, the complement of the second template sequence, the complement of the second identifier sequence, the complement of the fifth adaptor sequence, and the complement of the fourth adaptor sequence, wherein a sequencing primer is capable of hybridizing to the complement of the fifth adaptor sequence.

111. The method of claim 110, wherein the method further comprises contacting the first set of particles attached to a first polynucleotide population with a blocking primer, wherein the blocking primer is capable of hybridizing to the complement of the fourth adaptor sequence; and optionally further comprising contacting the second set of particles attached to a second polynucleotide population with a blocking primer, wherein the blocking primer is capable of hybridizing to the complement of the fourth adaptor sequence.

112. The method of claim 110, wherein the method further comprises:
a) contacting the first set of particles attached to a first polynucleotide population with an enrichment primer, wherein the enrichment primer comprises a sequence that is capable of hybridizing to the complement of the fifth adaptor sequence, and wherein the enrichment primer comprises a first member of a binding pair, under conditions suitable for hybridizing the enrichment primer to the fifth adaptor sequence;
b) contacting the enrichment primer with a second member of the binding pair bound to a solid support; and
c) removing unbound components of the reaction mixture, thereby producing a first set of particles attached to a first enriched polynucleotide population; and optionally further comprising:
d) contacting the second set of particles attached to a second polynucleotide population with an enrichment primer, wherein the enrichment primer comprises a sequence that is capable of hybridizing to the complement of the fifth adaptor sequence, and wherein the enrichment primer comprises a first member of a binding pair, under conditions suitable for hybridizing the enrichment primer to the fifth adaptor sequence;
e) contacting the enrichment primer with a second member of the binding pair bound to a solid support; and
f) removing unbound components of the reaction mixture, thereby producing a second set of particles attached to a second enriched polynucleotide population.

113. The method of claim 112, further comprising contacting the first set of particles attached to a first enriched polynucleotide population and/or the second set of particles attached to a second enriched polynucleotide population with a blocking primer, wherein the blocking primer is capable of hybridizing to the complement of the fourth adaptor sequence.

114. The method of claim 110 wherein the first template polynucleotide is formed by fragmenting target polynucleotides isolated from a biological fluid, cell culture, or solid tissue, and attaching a second adaptor sequence, a first identifier sequence, and a first adaptor sequence to the fragmented target polynucleotides; and optionally the second template polynucleotide is formed by fragmenting target polynucleotides isolated from a biological fluid, cell culture, or solid tissue, and attaching a second adaptor sequence, a second identifier sequence, and a third adaptor sequence to the fragmented target polynucleotides.

115. The method of claim 110, wherein the first set of particle and the second set of particles are microparticles or beads.

116. The method of claim 110, wherein the second adaptor sequence comprises at least one universal sequence.

117. The method of claim 110, wherein the first identifier sequence is a first barcode sequence and the second identifier sequence is a second barcode sequence.

118. The method of claim 110, wherein the nucleic acid amplification condition comprises a primer extension reaction.

119. The method of claim 110, wherein the nucleic acid amplification condition comprises an isothermal amplification condition or a thermocycling amplification condition.

120. The method of claim 110, wherein the reaction mixture comprises a polymerase and a plurality of nucleotides.

121. The method of claim 120, wherein the reaction mixture comprises a recombinase and optionally at least one recombinase accessory protein, at least one recombinase loading protein and/or at least one single-stranded binding protein

122. The method of claim 121, wherein the recombinase is uvsX and optionally at least one recombinase loading protein is uvsY and/or at least one single-stranded binding protein is gp32.

123. The method of claim 110, wherein the reaction mixture is contained in a water droplet in an oil and water emulsion.

124. The method of claim 110, wherein the reaction mixture is part of a single continuous liquid phase that does not provide compartmentalization.

125. A method for enriching template polynucleotides, comprising:
a) providing a mixture of polynucleotides which include (i) a plurality of template polynucleotides and (ii) a plurality of primer dimer byproducts, wherein individual template polynucleotides from the plurality of template polynucleotides include, in a 5′ to 3′direction, a first universal adaptor sequence, a template sequence, a second universal adaptor sequence, and a third universal adaptor sequence, wherein individual primer dimer byproducts from the plurality of primer dimer byproducts include, in a 5′ to 3′ direction, a first universal adaptor sequence and a third universal adaptor sequence;
b) forming a plurality of pre-enrichment complexes by contacting the mixture of polynucleotides with a plurality of enrichment primers that are capable of hybridizing to at least a portion of the second universal adaptor sequence, wherein the enrichment primer includes an affinity moiety;
c) forming a plurality of blocked primer dimer complexes by contacting the mixture of polynucleotides with a plurality of blocking primers that are capable of hybridizing to at least a portion of the third universal adaptor sequence on the primer dimer byproducts; and
d) separating the plurality of pre-enrichment complexes from the plurality of blocked primer dimer complexes, thereby enriching the template polynucleotides and generating a plurality of enriched template polynucleotides, and
optionally amplifying the enriched template polynucleotides of step (d) to generate amplified enriched template polynucleotides.

126. The method of claim 125, wherein the mixture of polynucleotides in step (a) includes (i) a plurality of template polynucleotides attached to a support and (ii) a plurality of primer dimer byproducts attached to a different support, wherein individual template polynucleotides from the plurality of template polynucleotides include, in a 5′ to 3′direction, a first universal adaptor sequence, a template sequence, a second universal adaptor sequence, and a third universal adaptor sequence, and wherein the 5′ or 3′ end of the individual template polynucleotides are attached to the support.

127. The method of claim 125, where the enrichment primer includes an affinity moiety which selectively binds a receptor moiety.

128. The method of claim 125, wherein the plurality of pre-enrichment complexes are separated from the plurality of blocked primer dimer complexes by contacting the affinity moiety on the enrichment primers with a purification bead that is attached to one or more receptor moieties, to form an enrichment complex; wherein the enrichment complex is separated or removed from the plurality of blocked primer dimer complexes, thereby generating an enriched population of template polynucleotides.
